(19) United States
(12) Patent Application Publication
(10) Pub. No.: US 2023/0398332 A1
(43) Pub. Date: Dec. 14, 2023

(54) GUIDEWIRE SAFETY DEVICE

(71) Applicants: JEB Technologies Limited, Mildenhall (GB); Andrew HINDMARSH, Ely, Cambridgeshire (GB); Andrew James JOHNSTON, Cambridge, Cambridgeshire (GB)

(72) Inventors: Andrew Hindmarsh, Ely, Cambridgeshire (GB); Andrew James Johnston, Cambridge, Cambridgeshire (GB); Fisher Michael, Mildenhall, Suffolk (GB); Adam Licence, Mildenhall, Suffolk (GB); Sean Licence, Mildenhall, Suffolk (GB)

(21) Appl. No.: 18/033,828

(22) PCT Filed: Oct. 29, 2021
(86) PCT No.: PCT/GB2021/052819

§ 371 Date: Apr. 26, 2023

(30) Foreign Application Priority Data
Oct. 29, 2020 (GB) 2017155.9

(51) Int. Cl. A61M 25/09

(52) U.S. Cl. CPC A61M 25/09041; A61M 2025/09125; A61M 2025/09116; A61M 25/09

(57) ABSTRACT

A guide wire safety device for preventing retained guidewire events comprises a mainbody component located between a Luer hub component for connection to a Luer hub and a locking sleeve component for connection to a catheter, wherein the main body component houses a means for gripping a guidewire.

The present invention relates to a guidewire safety device. The device has particular application in preventing retained guidewire events.

### BACKGROUND OF THE INVENTION

When treating patients having a variety of medical conditions it is often necessary to insert a catheter through the patient's skin into a blood vessel or a body cavity to facilitate delivery of treatment (drugs via a catheter into a central vein, draining of an abscess in the abdomen or chest etc), and/or achieve accurate monitoring of the patient's physiology (central venous pressure measurements, arterial pressure measurements, cardiac output measurement etc).

The standard procedure for inserting this sort of catheter is a known as the Seldinger technique. In this procedure a needle is used to puncture the target blood vessel or body cavity, and a soft ended guidewire threaded through the needle into the blood vessel or body cavity so that part of the guidewire is in the patient, and the remaining part of the guidewire is outside the patient. The needle is then withdrawn over the guidewire leaving the guidewire in the blood vessel or body cavity. A catheter is then threaded over the wire into the blood vessel or body cavity. Finally, the guide wire is withdrawn, leaving the catheter in the desired body location. The Seldinger technique is shown in the FIG. 23 (courtesy of Shah R and www.slideshare.net).

Although the Seldinger technique is the ‘gold standard’ for percutaneous insertion of a catheter into a blood vessel or body cavity, the procedure is associated with a risk of guidewire retention in the patient which can cause serious complications. In the case of central venous catheter insertion, retained guidewires have been reported to cause cardiac arrhythmias, thrombosis, cardiac perforation and cardiac tamponade, with a reported mortality rate of up to 20% (1, 2). This is a commonly performed procedure with over 5 million central venous catheters inserted per annum in the US, and an estimated incidence of retained guidewire events of approximately 1 in every 3000 procedures (3).

Given the serious consequences, retained guidewire events have been designated as a “Never Event” in both the US and UK. A “Never Event” is a serious, preventable patient safety incident that should not occur. Despite this, there were 30 reports of retained guidewires following Seldinger procedures in the UK between 1 Apr. 2019 and 29 Feb. 2020, of which 14 were following insertion of a central venous catheter (4).

The primary reason for retained guidewire events following catheter insertion using the Seldinger technique is human error. The literature describes operator inattention, distraction, and inexperience, together with inadequate supervision, high workloads, and staff fatigue as potential causes (5-7). Despite systems being put in place to address these issues such as better training, adequate supervision, checklists and equipment checks following the procedure, the incidence of retained guidewires continues to rise. This is largely because removal of the guidewires still relies on the human operator to remember to do so, and solutions that rely solely on the operator preventing mistakes are unlikely to be completely effective.

Retained guidewires during the central venous catheter placement occur at a “critical point” in the Seldinger procedure. This is when the catheter is placed over the guidewire (8). It is at this point that clinicians can forget that the guidewire has not been removed and, without realizing this, continue to advance the catheter and finish the procedure by securing the catheter in place. Retention of the guidewire is then only discovered on or after the initial check X-ray. It is likely that the ‘critical point’ for guidewire retention during other types of Seldinger procedures is the same as for central venous catheter insertion.

A number of strategies have been developed and/or proposed to reduce or prevent retained guidewire events. Based on the concept of safety engineering, these have focused on either amending the Seldinger procedure by introducing additional steps to increase safety, or modifying the equipment used to perform catheter insertion.

For example, Venner's WireSafe is an engineered solution which prevents completion of the central venous catheter insertion procedure without removing the guidewire. This consists of a locked box which contains the suture, suture holder, and antimicrobial dressing required to secure the central venous catheter. The box can only be opened using the guidewire as a key. It is inserted into the lock, and remaining inside the lock, is used as a handle to open the lid of the locked pack to allow the operator to access the suture, suture holder, and antimicrobial dressing. Therefore, the only way in which the operator can access the contents to complete the procedure is by first removing the guidewire from the patient after the “critical point”. This ensures that clinicians remember to remove the guidewire because they are unable to complete the procedure without doing so.

However, Venner's WireSafe suffers from the problems that it introduces additional steps to the Seldinger procedure which adds time to the procedure, and an additional step for clinicians. It still relies on the operator to adhere to the procedure protocol and has therefore not completely removed the element of human error from the procedure. It would not prevent guidewire retention in a clinical situation where two or more catheter packs are opened. This happens in two scenarios. The first is where there is a planned insertion of multiple central venous catheters within the same procedure, with the use of multiple kits. The second is where there has been difficulty in catheter insertion with the first kit, and a second kit is opened. Venner's WireSafe also has considerable cost implications.

There is no known equipment, or modifications of equipment, that is commercially available and designed to prevent retained guidewires.

However, a number of patent applications have been filed. Broadly these are divided into 2 groups:

Firstly, there are patent applications which describe different ways the guidewire could be modified to prevent its retention. These include different shaped wires, and wires that have some form of impediment structure such as a collapsible shape along its length, both of which would theoretically prevent migration of the guidewire into the patient if it was retained in the catheter. However, modifying the shape of the guidewire presents usability and safety issues because a bend in a guidewire is likely to make the wire more difficult to handle during a surgical procedure. In addition, a bend in the wire such as spiral is likely to result in the end of the guidewire rotating as the catheter is advanced over the wire. The rotating end of the wire is in the patient, and rotation of the guidewire tip could damage surrounding tissues and cause significant complications.

Secondly, there are patent applications which describe other locking mechanisms. For example, U.S. Pat. No. 8,992,480 describes a locking mechanism on the end of a central venous catheter, and outlines a number of different designs for achieving this. The document discloses a locking mechanism which must be activated by the operator i.e., with finger compression or other means. However, this manual control of the locking mechanism enables bidirectional travel of the guidewire through their device, and relies on the operator to activate the device to lock the guidewire in place.

In view of the above, there is a need for a new guidewire safety device which will assist in negating retained guidewire events.

Remarkably, this has now been achieved by a device incorporating a guidewire locking mechanism at the external end of a catheter. The device is attached (by the manufacturer of a catheter) to the end of the catheter channel that transmits the guidewire during insertion, but detachable from the catheter once a surgical procedure has been completed.

### SUMMARY OF THE INVENTION

In accordance with a first aspect of the present invention there is provided a guide wire safety device which comprises a main body component located between a Luer hub component for connection to a Luer hub and a locking sleeve component for connection to a catheter, wherein the main body component houses a means for gripping a guidewire.

Preferably, the means for gripping a guidewire includes a gripper. Preferably, the gripper allows free movement of a guidewire outwards towards the Luer hub component from the locking sleeve component, but it grips the guidewire preventing movement of the guidewire through the device in the opposite direction.

Preferably, the gripper is biased to grip a guidewire without requiring operator intervention if the guidewire is pulled or pushed through the device from the Luer hub towards the locking sleeve component. In addition, preferably, the gripper is biased to release the guidewire without requiring operator intervention if the guidewire is pulled or pushed through the device from the locking sleeve component towards the Luer hub.

Preferably, the gripper is selected from a collet, one or more barbs, a clamp, a clamp ball, a grip washer, a steel insert, a cam lock, an o-ring, a duckbill and a wedge. Most preferably, the gripper is a clamp ball.

Preferably, a single gripper is provided. For example, one embodiment of the invention includes only one clamp ball.

Preferably, the main body is manufactured by injection moulding or ultrasonic welding of a plastics material. Preferably, the plastics material is selected from ABS, polypropylene or polycarbonate. Preferably, it is colourless or clear. Advantageously, this allows the user to have full visibility of the guidewire and procedural fluids.

Preferably, the main body has length of about 20 mm to about 25 mm.

Preferably, the main body has a maximum outside diameter of about 8 mm.

Preferably, the main body has an internal empty volume of about 20 mm³ to about 136 mm³.

Preferably, an end of the main body adjacent to the locking sleeve component has a shallow external inclusive angle about 3.4° of the outside profile. Advantageously, this provides a secure leak free taper fit to a mating catheter.

Preferably, the Luer hub component is manufactured by injection moulding plastics materials such as polypropylene, polycarbonate or ABS.

Preferably, the Luer hub component has a length of about 10 mm to about 15 mm.

Preferably, the Luer hub component has a maximum outside diameter of about 8 mm.

Preferably, the Luer hub component is bonded to the main body component by either press fit retention, or using one or more bonding adhesives.

Preferably, the Luer hub component has an end for connection to a syringe. In this regard, preferably, the Luer hub component has an internal taper feature (which conforms to syringe manufacturing standards) that allows connection to a standard syringe, and/or a locking screw feature on the top of the Luer hub component (conforming to syringe manufacturing standards) which allows a syringe to be screwed to the component. Preferably, the Luer Hub component is compatible with both locking and slip style syringes.

Preferably, the Luer hub component has a lumen having a diameter of about 0.8 mm to about 1 mm, which is greater than that of the guide wire which allows for free unrestricted movement of the guide wire through the component.

The locking sleeve component is preferably manufactured by injection moulding plastic materials such as polypropylene, polycarbonate or ABS.

Preferably, the locking sleeve component has a length of about 10 mm to about 15 mm, more preferably about 12.7 mm.

Preferably, the locking sleeve component has a maximum outside diameter of about 10 mm to about 12 mm, more preferably 11 mm.

Preferably, the locking sleeve component is press fitted on to the main body component, and is preferably retained by a barb feature which is present on the main body component.

Preferably, the locking sleeve component moves freely in a rotational direction around the main body. Advantageously, this allows for securing to the catheter without causing rotation movement of the assembly.

Preferably, an internal thread is present in the internal bore of the locking sleeve component (manufactured in line with syringe manufacturing standards) which allows for a secure screw connection to a catheter.

Advantageously, the gripper allows the guidewire to move freely through it in one direction, but automatically grips and locks the guidewire if it is moved in the opposite direction. This allows the catheter to be threaded over the guidewire and freely advanced into the blood vessel or body cavity in the patient. It also allows withdrawal of the guidewire from the catheter and out of the patient once the catheter is in position. However, once the external end of the guidewire has been threaded through the gripper, it is not possible for the guidewire to be accidentally pushed back into the patient, nor would it be able to migrate into the patient if accidentally left in the lumen of the catheter at the end of the procedure.

The invention provides the advantages of an engineered safety solution thereby removing the potential for human operator error as the locking mechanism activates automatically. The device of the invention is relatively easy to engineer and manufacture; it has a low cost; it is safe in scenarios when multiple catheter packs are open; it has a single locking mechanism design and size can be used with all current diameters of central venous catheter guidewire; and the device can be modified for use on all catheters (not just central venous catheters) inserted using the Seldinger technique to prevent guidewire retention events.

Preferably, the gripper allows only unidirectional travel of the guidewire.

Preferably, the gripper locks automatically if the guidewire is moved in the other direction. This provides the advantage of removing any reliance on the operator prevent guidewire retention i.e. removing human error.

Preferably, the gripper effectively locks central venous catheter guidewires having different diameters without any modification to the gripper.

Preferably, the gripper effectively locks the guidewire regardless of orientation.

Preferably, the gripper will effectively lock the guidewire even if the guidewire or gripper are coated in saline, blood or other body fluids.

Preferably, the gripper can be modified (scaled) to effectively lock any guidewire used in any Seldinger procedure, not just central venous catheter insertion.

Preferably, the device of the invention includes a gripper which is capable of releasing the lock on the guidewire.

Preferably, the device defines at least one lumen which extends through the main body component, Luer hub component and locking sleeve component for allowing flushing fluid through the device. The lumen allows free passage of fluids through the device. This provides the advantage of enabling a catheter with the device attached to be used to administer fluids, or drain effluent. In addition, in the event that an operator forgets to remove the device once a catheter or drain has been put in place it provides the advantage of mitigating risk to the patient.

In one embodiment, preferably the lumen comprises a plurality of fenestrations between the gripper and an inner wall of the main body.

The present invention provides the advantage of allowing only unidirectional wire movement. In all embodiments of the invention, movement of the wire into the patient is prevented by a self-activating and self-releasing mechanism which is triggered by movement of the wire itself. No separate mechanical switch is required.

The invention provides specific fenestrations/channels that allow each embodiment of the invention to be flushed before use.

### BRIEF DESCRIPTION OF THE DRAWINGS

The invention will now be further described with reference to the accompanying drawings in which:

FIG. 1 shows a view of a first embodiment of the invention, FIG. 1A shows a front view, FIG. 1B shows a section view of the device wherein the griper is gripped onto the guide wire, and FIG. 1C shows a section view of the device wherein the gripper is ungripped from the guide wire;

FIG. 2 shows a further section view of this embodiment without a clamp ball or guide wire enabling the channels through the device to be seen clearly;

FIG. 3 shows a cross section view of this embodiment showing a guide wire is located in a guidewire channel and a clamp ball is located in a clamp ball channel;

FIG. 4 shows a further cross section of this embodiment showing adjoining channels for a guidewire and a clamp ball.

FIG. 5 shows a view of a second embodiment of the invention, FIG. 5A shows a front view, FIG. 5B shows a section view of the device wherein the griper is gripped onto the guide wire, and FIG. 5C shows a section view of the device wherein the gripper is ungripped from the guide wire;

FIG. 6 shows a sectional view of this embodiment showing the ramp face is tapered at an angle of about 16°;

FIG. 7 shows a sectional view of a collet of this embodiment;

FIG. 8 shows a further sectional view of an alternative collet of this embodiment;

FIG. 9 shows a further sectional view of a collet of this embodiment showing the activation radius of the collet;

FIG. 10 shows a view of a third embodiment of the invention, FIG. 10A shows a front view, FIG. 10B shows a section view of the device wherein the griper is gripped onto the guide wire, and FIG. 10C shows a section view of the device wherein the gripper is ungripped from the guide wire;

FIGS. 11 shows an alternative view of the barbs of this embodiment;

FIG. 12 shows an alternative view of this embodiment showing fenestrations through the device;

FIG. 13 shows an alternative view of this embodiment showing it as a single moulding;

FIG. 14 shows a view of a fourth embodiment of the invention, FIG. 14A shows a front view, FIG. 14B shows a section view of the device wherein the griper is gripped onto the guide wire, and FIG. 14C shows a section view of the device wherein the gripper is ungripped from the guide wire;

FIG. 15 shows a view of a fifth embodiment of the invention, FIG. 15A shows a front view, FIG. 15B shows a section view of the device wherein the griper is gripped onto the guide wire, and FIG. 15C shows a section view of the device wherein the gripper is ungripped from the guide wire;

FIG. 16 shows a cross section of this embodiment showing a single channel for a guidewire and a clamp ball;

FIG. 17 shows a view of a sixth embodiment of the invention, FIG. 17A shows a front view, FIG. 17B shows a section view of the device wherein the griper is gripped onto the guide wire, and FIG. 17C shows a section view of the device wherein the gripper is ungripped from the guide wire;

FIG. 18 shows a view of a seventh embodiment of the invention, FIG. 18A shows a front view, FIG. 18B shows a section view of the device wherein the griper is gripped onto the guide wire, and FIG. 18C shows a section view of the device wherein the gripper is ungripped from the guide wire;

FIG. 19 shows a view of a eighth embodiment of the invention, FIG. 19A shows a front view, FIG. 19B shows a section view of the device wherein the griper is gripped onto the guide wire, and FIG. 19C shows a section view of the device wherein the gripper is ungripped from the guide wire;

FIG. 20 shows a view of a ninth embodiment of the invention, FIG. 20A shows a front view, FIG. 20B shows a section view of the device wherein the griper is gripped onto the guide wire, and FIG. 20C shows a section view of the device wherein the gripper is ungripped from the guide wire;

FIG. 21 shows a view of a tenth embodiment of the invention, FIG. 21A shows a front view, FIG. 21B shows a section view of the device wherein the griper is gripped onto the guide wire, and FIG. 21C shows a section view of the device wherein the gripper is ungripped from the guide wire;

FIG. 22 shows a view of a eleventh embodiment of the invention, FIG. 22A shows a front view, FIG. 22B shows a section view of the device wherein the griper is gripped onto the guide wire, and FIG. 22C shows a section view of the device wherein the gripper is ungripped from the guide wire; and

FIG. 23 shows the known Seldinger technique.

### DETAILED DESCRIPTION OF THE INVENTION

It will be appreciated that aspects, embodiments and preferred features of the invention have been described herein in a way that allows the specification to be written in a clear and concise way. However, unless circumstances clearly dictate otherwise, aspects, embodiments and preferred features can be variously combined or separated in accordance with the invention. For example, features of the second and third embodiments described below can be combined. In this regard, for example, a collet mechanism can be combined with barbed arms at the collet/wire interface. This can reduce the force needed to push the wire through the device in the non-locking direction as a larger central hole through the collet can be provided. A combination of barbs gripping the wire and a collet being pulled back into a ramped part of a housing so that the collet exerts a radially inward force on the wire results in stopping movement of the wire relative to the device in an inward direction (towards the patient).

Within the context of this specification, the word “about” means plus or minus 20%, more preferably plus or minus 10%, even more preferably plus or minus 5%, most preferably plus or minus 2%.

Within the context of this specification, the word “comprises” means “includes, among other things” and should not be construed to mean “consists of only”.

Within the context of this specification, the word “substantially” means preferably at least 90%, more preferably 95%, even more preferably 98%, most preferably 99%.

Within the context of this specification, the term “gripper” is used interchangeably with the term “locking mechanism”.

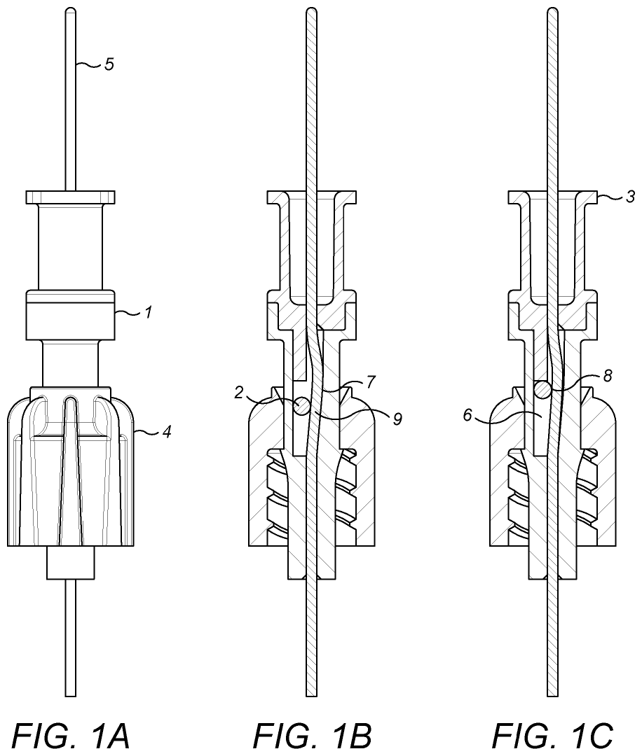

With reference to FIGS. 1 to 4, in one embodiment, the guide wire [5] is restricted from moving in the inwards direction by progressively increasing the clamping force provided by a clamp ball on the guide wire via a taper activation method.

According to this embodiment, the device is designed to function to its intended use in all orientations including, upright, lay flat or inverted, positions as well as any other orientation in between. As an assembled product, the device is about 31 mm in length and securely connects to the Luer hub of a standard Catheter. The device has a central lumen which allows free and unrestricted movement of a guidewire through the centre of the device in one direction (free movement), but automatically grips and locks the guidewire in place preventing movement of the guidewire through the device in the opposite direction (lock). This gripping effect automatically releases on changing the direction of the guidewire movement to outwards. In both the configurations of the embodiment (free movement or locked), the design of the device enables bidirectional free flow of fluid through the device.

The main body [1] component is preferably manufactured by injection moulding, or ultrasonic welding, of plastics materials such as ABS, polypropylene or polycarbonate. Preferably, the main body component is colourless or clear. Preferably, it has a length of about 22 mm and a maximum outside diameter of about \acute{Ø}10 mm. Preferably, the main body component [1] is translucent, more preferably colourless. Preferably, the main body component [1] has an internal empty volume [6] about 38.88 mm³ which allows a user to have full visibility of the guidewire and procedural fluids though the main body component [1]. The main body component also has an internal shallow convex radius [7] of between about 10 mm and 50 mm that forms a ramp feature necessary to stop movement of the guide wire towards a patient relative to the device via a direct interaction between the ramp feature and the guidewire, when a force is exerted on the guidewire by the clamp ball. The clamp ball and guidewire are each located in separate adjoined channels [10], [11] that advantageously prevent the clamp ball entering the guide wire channel.

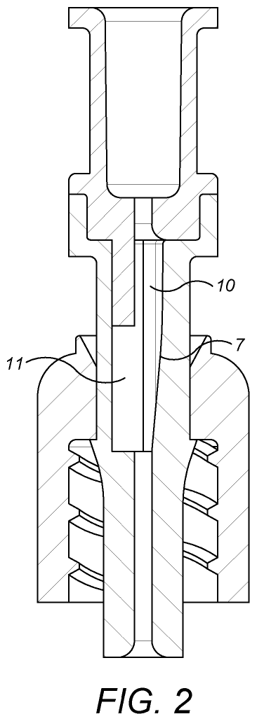

FIG. 2 shows the device without a clamp ball or guide wire enabling the channels [10], [11] to be seen clearly.

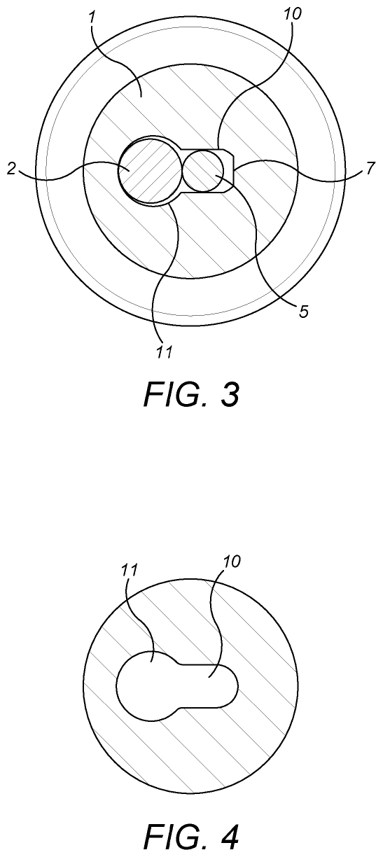

As shown in FIG. 3, preferably the guide wire is located in guidewire channel [10] and the clamp ball is located in clamp ball channel [11]. The clamp ball channel has an adequate size to allow the clamp ball to axially travel along the length of the clamp ball channel. These adjoined channels may have a circular, ovoid or a polygon shape, or any combination of these. The open interface between the two channels has a width of between about 0.5 mm to about 2 mm.

As shown in FIG. 4, the guide wire channel [10 ] and clamp ball channel [11] have an open interface.

The ramp feature may have a smooth or textured surface finish. For example, the texture can be provided by projections and/or indents in the surface of the ramp feature. Advantageously, a textured surface finish promotes friction between the ramp and guide wire. At the lower end of the main body, the body is tapered. A shallow external inclusive angle of about 3.44° of the outside profile is manufactured to provide a secure leak free taper fit to the mating catheter.

The clamp ball component [2] is preferably manufactured from plastic materials such as polypropylene, polycarbonate and ABS, or stainless 304 or 316 materials. Preferably a single clamp ball is provided. The clamp ball is housed inside the main body of the device, where it is free to move within a channel [11] in the main body. The clamp ball has an external diameter of about 1 mm to about 3 mm and remains in contact with the guide wire in both the locked and unlocked configurations. The clamp ball has a smooth or textured surface finish to enhance friction with the guide wire. For example, the texture can be provided by projections and/or indents in the surface of the clamp ball.

The surface of the clamp ball may be textured to promote friction between the clamp ball, the guidewire and the convex ramp feature.

In use, when the guidewire is pushed or pulled through the male component on the Luer hub in an outwards direction (away from the patient relative to the device) it is directed through a channel though the main body. The clamp ball lightly grips the guidewire via its critically controlled outside diameter but does not provide any resistance to wire movement [8]. In contrast, if the guidewire is pushed in an inwards direction (towards the patient relative to the device), the critically controlled outside diameter of the clamp ball means that the clamp ball lightly grips the guidewire and the guidewire is pulled into the ramp of the main body of the device. On contact with the ramp feature of the main body of the device, the shallow radius of the main body of the ramp feature and the clamp ball component combine to tighten the grip of the clamp ball on the guidewire and this stops movement of the guidewire in the inward direction relative to the device [9].

The Luer hub [3] component is preferably manufactured by injection moulding a plastics material. Preferably the plastics material is selected from polypropylene, polycarbonate or ABS. Preferably, it has a length of about 15 mm and a maximum outside diameter of about 10 mm is bonded to the main body component by either press fit retention, ultrasonic welding or using bonding adhesives. At its lower end, the Luer hub component has a channel therethrough having a diameter of about 1 mm to about 2 mm which is greater than that of the guide wire. This allows for free unrestricted movement of the guide wire through the Luer hub component. The Luer hub component has an internal taper feature (which conforms to syringe manufacturing standards) that allows connection to a standard syringe, and a locking screw feature on the top of the upper end of the Luer hub component (conforming to syringe manufacturing standards) which allows a syringe to be locked to the component. It is intentional that the Luer Hub component is compatible with both locking and slips style syringes.

The locking sleeve [4] component is preferably manufactured by injection moulding a plastics material. Preferably, the plastics material is selected from polypropylene, polycarbonate or ABS. Preferably, it has a length of about 12.7 mm and a maximum outside diameter of about 11 mm is press fitted on to the main body component, and is retained by a barb feature which is present on the main body component. The locking sleeve is able to move freely in a rotational direction around the main body allowing for securing to the catheter without causing rotation movement of the assembly. There is an internal thread present in the internal bore of the locking sleeve (manufactured in line with syringe manufacturing standards) which allows for secure screw connection to the catheter.

The device defines at least one lumen which extends through the main body component, Luer hub component and locking sleeve component for allowing flushing fluid through the device. The lumen allows free passage of fluids through the device. This provides the advantage of enabling a catheter with the device attached to be used to administer fluids, or drain effluent. In addition, in the event that an operator forgets to remove the device once a catheter or drain has been put in place it provides the advantage of mitigating risk to the patient.

Preferably, the lumen comprises a plurality of fenestrations in the main body.

Advantageously, the clamp ball effectively locks the guidewire and stops it from moving in an inward direction (towards the patient) even if the guidewire or clamp ball are coated in saline, blood or other body fluids.

Preferably, this embodiment comprises only one clamp ball.

Preferably, this embodiment defines separate channels through the device, one channel [10] for the guidewire and a separate channel [11] for the clamp ball. This provides the advantage of preventing the clamp ball from obstructing the passage of the guidewire through the device. In addition, the separate channels provide the advantage that the clamp ball moves freely within its recessed channel and is always in contact with the guidewire.

The device incorporates a specific taper on the guidewire channel, namely a convex curved internal wall, which improves the contact and force between the walls, the ball, and the wire.

Advantageously, the surface of the ball and sidewalls of the channels in which the ball and guidewire are provided are configured to improve friction between the ball and wire and between the wire and sidewall.

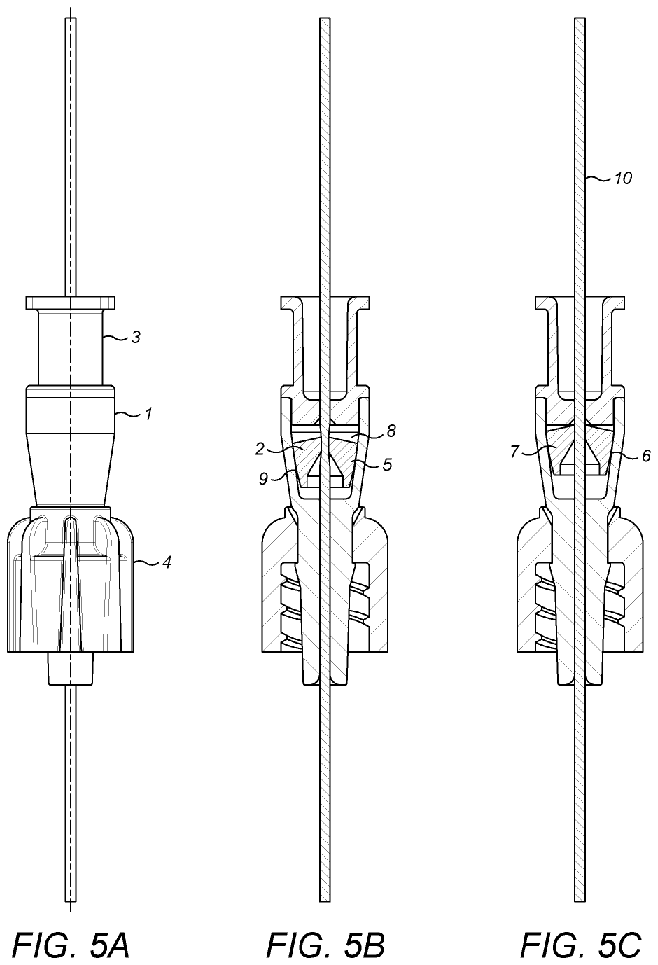

With reference to FIGS. 5 to 9, in a second embodiment, the guide wire [10] is restricted from moving in the inwards direction by progressively increasing the compression force acting on the guidewire via a collet and taper activation method.

According to this embodiment, the device is designed to function to its intended use in all orientations including, upright, lay flat or inverted, positions as well as any other orientation in between. As an assembled product, the device is about 34 mm in length and securely connects to the Luer hub of a standard Catheter. The device has a central lumen which allows free and unrestricted movement of a guidewire through the centre of the device in one direction (free movement), but automatically grips and locks the guidewire in place preventing movement of the guidewire through the device in the opposite direction (lock). This gripping effect automatically releases on changing the direction of the guidewire movement to outwards. In both the configurations of the collet (free movement or locked), the design of the device enables bidirectional free flow of fluid through the device.

The main body [1] component is preferably manufactured by injection moulding, or ultrasonic welding, of plastics materials such as ABS, polypropylene or polycarbonate. Preferably, the main body component is colourless or clear. Preferably, it has a length of about 25 mm and a maximum outside diameter of about 8 mm. The main body component has an internal empty volume [8] of about 136 mm³ which allows the user to have full visibility of the guidewire and procedural fluids. The main body component also has an internal shallow angle [9] which tapers at an inclusive angle of about 16° and forms a ramp feature necessary to activate the collet component. The ramp feature is preferably linear, concave, convex or a combination thereof. In the case of a concave or convex ramp feature, the radius of curvature is preferably about 10 mm to about 50 mm. Advantageously, by providing a ramp feature having a concave or convex face, a shorter clamping response time can be achieved. In a preferred embodiment, the ramp feature has a linear, conical ramp face on the inner wall of the main body.

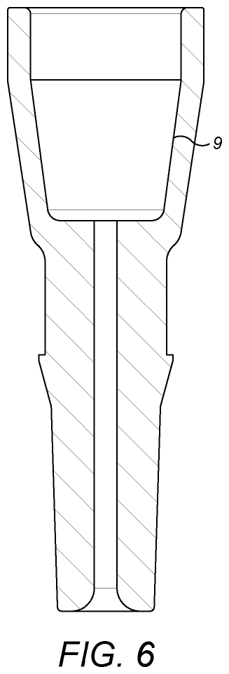

As shown in FIG. 6, preferably the ramp face [9] is tapered at an inclusive angle of about 16°. The radiused face of the collet abuts this ramp face and as the collet is forced to move longitudinally through the main body, force applied by the tapering ramp face of the inner wall of the main body forces causes the collet to grip the guide wire. At the lower end of the main body, the body has a shallow external inclusive angle of about 3.4° of the outside profile is manufactured to provide a secure leak free taper fit to the mating catheter.

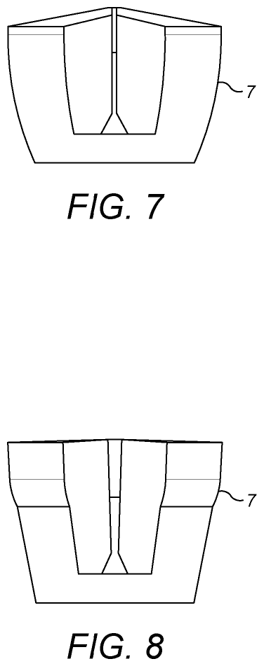

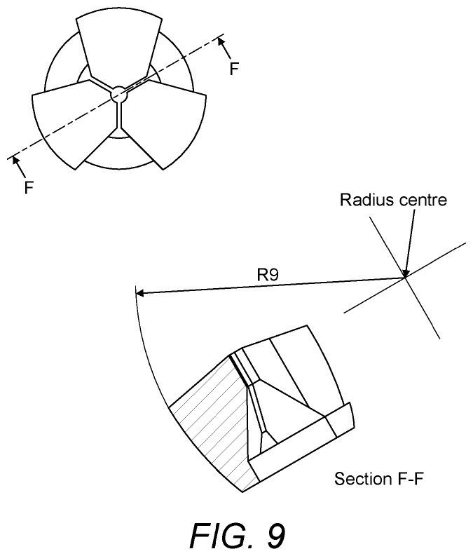

The collet [2] component is manufactured by injection moulding plastic materials such as polypropylene, polycarbonate and ABS, and is housed in a specific orientation inside the main body of the component, where it will be free to move in a forward and backward direction during use. The collet has a length of about 4.2 mm and a maximum outside diameter of about 5.8 mm and has preferably 3 to 5, more preferably 3, leg features equally spaced in a round construction, separated by preferably 3 to 5, more preferably 3, empty spaces of similar size and connected to each other at one or other end of the collet component. A hole of equal or smaller diameter to the specific guidewire, and with a diameter of about 0.5 mm is present through the centre of the component. As shown in FIG. 7, preferably, the outside profile of the collet, has an activation radius [7] of about 9 mm. Alternatively, as shown in FIG. 8, preferably, the outside profile of the collet, has an activation radius [7] of about 2 mm. As shown in FIG. 9, the activation radius is shown as R9 described from the radius centre.

When the guidewire is pushed or pulled through the male component on the Luer hub in an outwards direction (away from the patient) it is directed through the central hole of the collet. The collet lightly grips the guidewire [5] via its critically controlled central hole but does not provide any resistance to wire movement. In contrast, if the guidewire is pushed in an inwards direction (towards the patient), the critically controlled central hole means that the collet will lightly grip the guidewire and be pulled into the ramp of the main body of the device. On contact with the ramp feature of the main body of the device, the angle of the main body of the ramp feature and the radius of the collet component combine to further close the central hole of the collet. This closing effect tightens the grip of the collet on the guidewire and stops the movement of the guidewire in the inward direction [6].

The Luer hub [3] component is preferably manufactured by injection moulding a plastics material. Preferably the plastics material is selected from polypropylene, polycarbonate or ABS. Preferably, it has a length of about 11 mm and a maximum outside diameter of about 8 mm is bonded to the main body component by either press fit retention, or using bonding adhesives. At its lower end, the Luer hub component has a hole diameter of about 1 mm to about 2 mm, which is greater than that of the guide wire which allows for free unrestricted movement of the guide wire through the Luer hub component. The Luer hub component has an internal taper feature (which conforms to syringe manufacturing standards) that allows connection to a standard syringe, and a locking screw feature on the top of the upper end of the Luer hub component (conforming to syringe manufacturing standards) which allows a syringe to be locked to the Luer hub component. It is intentional that the Luer Hub component is compatible with both locking and slip style syringes.

The locking sleeve [4] component is preferably manufactured by injection moulding a plastics material. Preferably, the plastics material is selected from polypropylene, polycarbonate or ABS. Preferably, it has a length of about 12.7 mm and a maximum outside diameter of about 11 mm is press fitted on to the main body component, and is retained by a barb feature which is present on the main body component. The locking sleeve is able to move freely in a rotational direction around the main body allowing for securing to the catheter without causing rotation movement of the assembly. There is an internal thread present in the internal bore of the locking sleeve (manufactured in line with syringe manufacturing standards) which allows for secure screw connection to the catheter.

The device defines at least one lumen which extends through the main body component, Luer hub component and locking sleeve component for allowing flushing fluid through the device. The lumen allows free passage of fluids through the device. This provides the advantage of enabling a catheter with the device attached to be used to administer fluids, or drain effluent. In addition, in the event that an operator forgets to remove the device once a catheter or drain has been put in place it provides the advantage of mitigating risk to the patient.

The lumen comprises a plurality of fenestrations between the collet and an inner wall of the main body.

Advantageously, the collet effectively locks the guidewire even if the guidewire or collet are coated in saline, blood or other body fluids.

In this embodiment, a single mobile element (the collet) is located within a fixed main body. That is to say that the collet is movable in relation to the main body. Interactions between the inner wall of the main body and the outer wall of the collet result in inward radial forces that alter the force between the inner wall of the collet and a guidewire. This force is biphasic in that it automatically increases if the wire is moved towards the patient, gripping the wire and so preventing movement in a direction towards the patient, but reduces when the wire is moved out of the patient and allows free movement of the wire. The embodiment requires multiple features working together to allow this automated release and grip of the guidewire. These include:
a. The collet defines a central aperture that allows passage of wire whilst always maintaining ‘light’ contact with the wire so that the collet always moves in the direction of the wire. The design of the central aperture and the inner wall of the collet is such that the wire is freely directed up the collet and can freely move within the collet, but that resistive forces between the inner wall and wire are maximised.
b. The inner wall of the main body and the outer wall of the collet are tapered (linear, concave, convex or any combination thereof) in the direction of the patient. This provides the advantage of causing the collet to grip the wire when the wire is moved towards the patient relative to the device.
c. The collet has a plurality of legs which are pivoted at one end or the other of the collet. Movement of the collet into the taper provides a radial force onto the legs which then deforms the collet to compress the guidewire within the central aperture—the strength of this force is proportional to the degree of movement of the collet into the taper. Movement of the collet out of the taper releases the legs and thus the guidewire. The stiffness and flexibility of the collet are designed to maximise the activation and release of these compressive forces.
d. Advantageously, the above mechanism is self-activating and self-releasing. No separate switch or mechanism are required.
e. Advantageously, the collet is fenestrated to allow fluids to pass therethrough and to allow the device to be flushable before use.

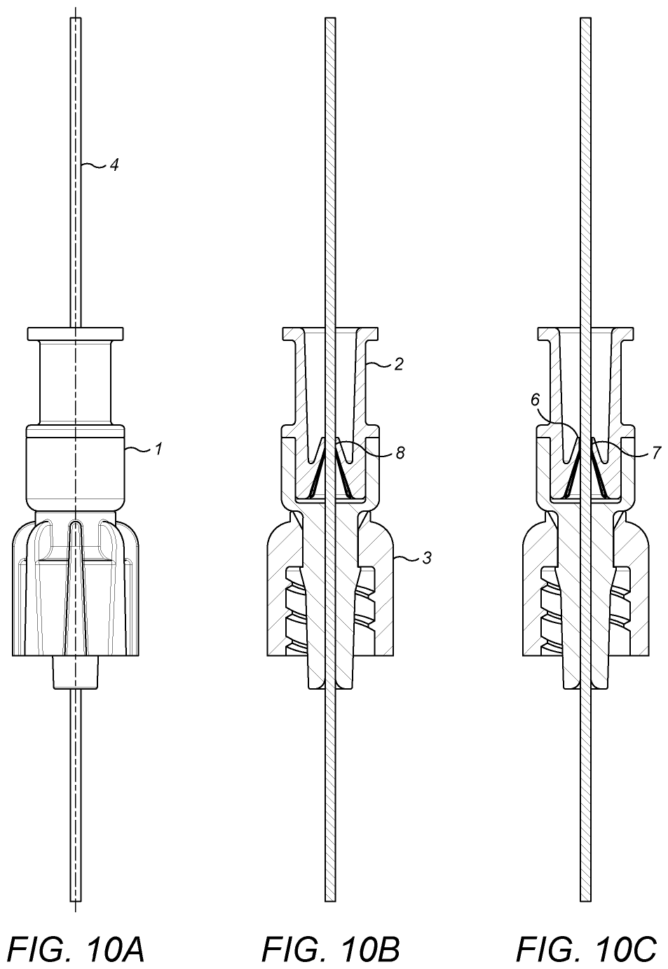

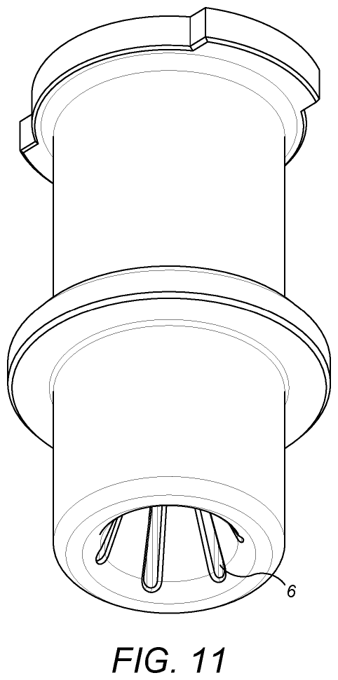

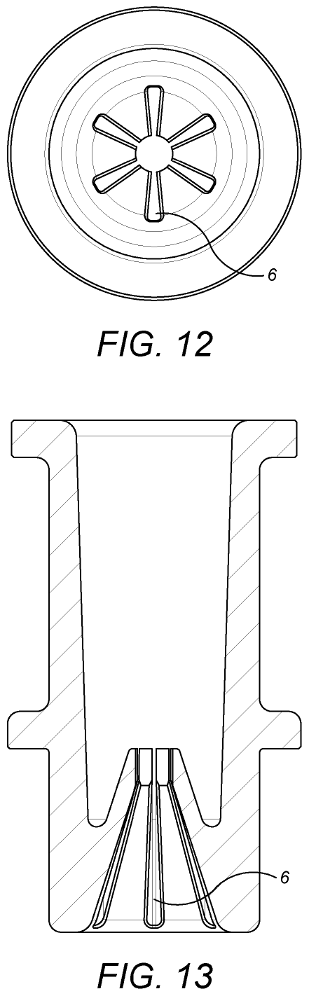

With reference to FIGS. 10 to 13, in a third embodiment, the guidewire [4] is restricted from moving in the inwards direction by the injection moulded protruding barbs biting into the guide wire.

The device is designed to function to its intended use in all orientations including, upright, lay flat or inverted, positions as well as any other orientation in between. As an assembled product, the device is about 30.7 mm in length and securely connects to the Luer hub of a standard Catheter. The device has a central lumen which allows free and unrestricted movement of a guidewire through the centre of the device in one direction (free movement), but automatically grips and locks the guidewire in place preventing movement of the guidewire through the device in the opposite direction (lock). This gripping effect automatically releases on changing the direction of the guidewire movement to outwards. In both the configurations of the barb features (free movement or locked), the design of the device enables bidirectional free flow of fluid through the device.

The main body [1] component is preferably manufactured by injection moulding, or ultrasonic welding, of plastics materials such as ABS, polypropylene or polycarbonate. Preferably, the main body component is colourless or clear. Preferably, it has a length of about 21.7 mm and a maximum outside diameter of about 8 mm. The main body component has an internal empty volume [5] of about 90 mm³ which allows the user to have full visibility of the guidewire and procedural fluids. At the lower end of the main body, the shallow external inclusive angle about 3.4° of the outside profile is manufactured to provide a secure leak free taper fit to the mating catheter.

The Luer hub [2] component is preferably manufactured by injection moulding a plastics material. Preferably the plastics material is selected from polypropylene, polycarbonate or ABS. Preferably, it has a length of about 14 mm and a maximum outside diameter of about 8 mm is bonded to the main body component by either press fit retention, or using bonding adhesives. As shown in FIGS. 11 to 13, at its lower end, the component preferably has about 4 to about 6 barbed features [6] set at about 50° to about 70° from the centre line of the component. The 4 or 6 barb features come together to create a hole of equal or smaller diameter to the specific guidewire and with a target hole diameter of about 0.8 mm. The barbs create a light grip on movement of the guidewire in the outwards direction, and these barbed features deflect out to allow free movement of the guide wire [7]. If the guidewire is pushed in an inwards direction (towards the patient), the barb features bite in to the guide wire which tightens the grip of the barbs on the guidewire and stops the movement of the guidewire in the inward direction [8]. The angle, shape, thickness and/or flexibility of the barbs is such that the optimum resistance is applied to the guidewire.

The Luer hub component has an internal taper feature (which conforms to syringe manufacturing standards) that allows connection to a standard syringe, and a locking screw feature on the top of the upper end of the component (conforming to syringe manufacturing standards) which allows a syringe to be locked to the component. It is intentional that the Luer Hub component is compatible with both locking and slip style syringes.

The locking sleeve [3] component is preferably manufactured by injection moulding a plastics material. Preferably, the plastics material is selected from polypropylene, polycarbonate or ABS. Preferably, it has a length of about 12.7 mm and a maximum outside diameter of about 11 mm is press fitted on to the main body component, and is retained by a barb feature which is present on the main body component. The locking sleeve is able to move freely in a rotational direction around the main body allowing for securing to the catheter without causing rotation movement of the assembly. There is an internal thread present in the internal bore of the locking sleeve (manufactured in line with syringe manufacturing standards) which allows for secure screw connection to the catheter.

The device defines at least one lumen which extends through the main body component, Luer hub component and locking sleeve component for allowing flushing fluid through the device. The lumen allows free passage of fluids through the device. This provides the advantage of enabling a catheter with the device attached to be used to administer fluids, or drain effluent. In addition, in the event that an operator forgets to remove the device once a catheter or drain has been put in place it provides the advantage of mitigating risk to the patient.

The lumen comprises a plurality of fenestrations between the barbed moulding and an inner wall of the main body.

Advantageously, the barbed moulding effectively locks the guidewire even if the guidewire or barbed moulding are coated in saline, blood or other body fluids.

In this embodiment, a plurality of barbs, preferably three or more, are positioned around a central channel through which a guidewire can pass. The barbs are directed away from the patient, and they always maintain light contact with the guidewire. The guidewire can move freely out of the patient but movement into the patient relative to the device results in a resistive radial force on the wire which prevents further movement. The mechanism is self-activating and releasing. The embodiment requires multiple features working together to allow this automated release and grip of the guidewire. These include:
a. Remarkably, the mechanism is made using a single moulding.
b. The barb angles, shapes, and flexibility are designed to maximise contact between the barbs and guidewire, and to provide adequate and rapid grip when the wire moves towards the patient relative to the device.
c. The design of the barbs is such that they cannot be inverted by normal clinical forces.
d. Advantageously, fenestrations between arms/barbs are provided to allow fluids to pass therethrough thereby allowing the device to be flushable before use.
e. When the wire is moved in one direction (against the arms/barbs) the arms/barbs grip the wire, and the stiffness of the material prevents inversion of the arms/barbs so the wire can't move. Advantageously, the barbs are self-activating. No separate switch or mechanism is required.
f. Movement of the wire relative to the device in the direction away from the patient opens (or everts or deflects out) the arms/barbs and the wire is released and can move freely. Advantageously, the barbs are self-releasing. No separate switch or mechanism is required.

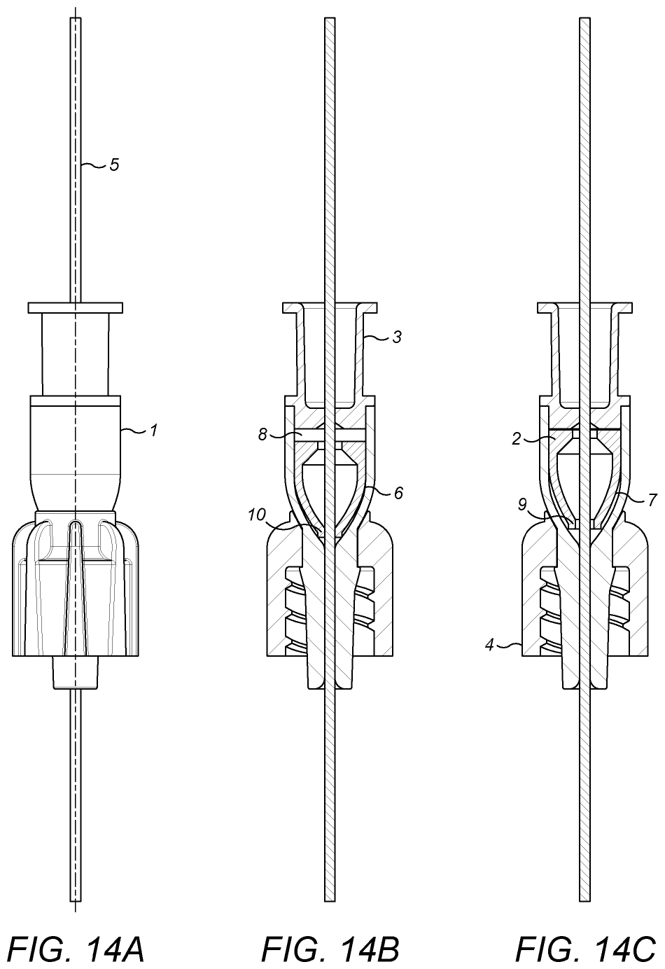

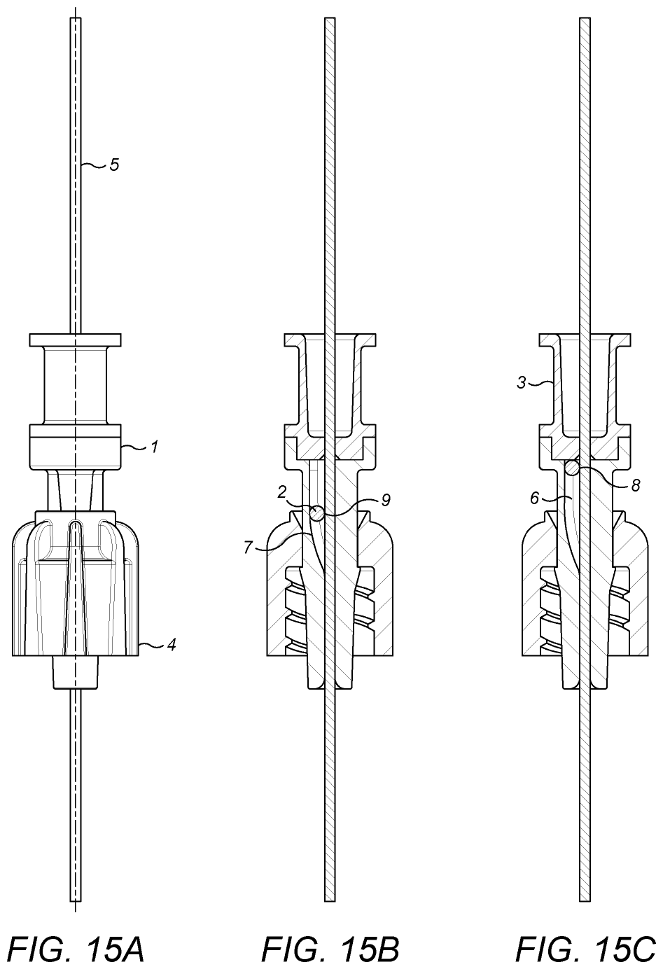

With reference to FIG. 14, in a fourth embodiment the guide wire [5] is restricted from moving in the inwards direction by progressively increasing the compression force acting on the guide wire via a taper activation method.

The device is designed to function to its intended use in all orientations including, upright, lay flat or inverted, positions as well as any other orientation in between. As an assembled product, the device is about 34 mm in length and securely connects to the Luer hub of a standard Catheter. The device has a central lumen which allows free and unrestricted movement of a guidewire through the centre of the device in one direction (free movement), but automatically grips and locks the guidewire in place preventing movement of the guidewire through the device in the opposite direction (lock). This gripping effect automatically releases on changing the direction of the guidewire movement to outwards. In both the configurations of the clamp (free movement or locked), the design of the device enables bidirectional free flow of fluid through the device.

The main body [1] component is preferably manufactured by injection moulding, or ultrasonic welding, of plastics materials such as ABS, polypropylene or polycarbonate. Preferably, the main body component is colourless or clear. Preferably, it has a length of about 25 mm and a maximum outside diameter of about 8 mm. The main body component has an internal empty volume [8] of about 126 mm³ which allows the user to have full visibility of the guidewire and procedural fluids. The main body component also has an internal Radius [6] of about 8.3 mm that forms the ramp feature necessary to activate the clamp component. At the lower end of the main body, the shallow external inclusive angle of about 3.4° of the outside profile is manufactured to provide a secure leak free taper fit to the mating catheter.

The clamp [2] component is manufactured by injection moulding plastic materials such as polypropylene, polycarbonate and ABS, and is housed in a specific orientation inside the main body of the component, where it will be free to move in a forward and backward direction during use. The clamp has a length of about 8.8 mm and a maximum outside diameter of about 6 mm and has at least 2 leg features equally spaced in a round construction, separated by empty spaces and connected to each other at the larger diameter end of the component. A critically controlled gap that is equal or smaller in distance than diameter of the specific guidewire, and with a gap dimension of about 0.2 mm is present through the centre of the clamp feature of the component. The outside profile of the clamp legs is equal or slightly greater in Radius [7] of about 8.1 mm to that of the internal Radius of the main body (ramp). The large diameter end of the clamp component has a hole diameter of about 2 mm which is greater than that of the guide wire and allows for free unrestricted movement of the guide wire through this section of the component.

When the guidewire is pushed or pulled through the male component on the Luer hub in an outwards direction (away from the patient) it is directed through the central hole of the clamp. The clamp lightly grips the guidewire via its critically controlled central hole but does not provide any resistance to wire movement. If the guidewire is pushed in an inwards direction (towards the patient), the critically controlled central hole means that the clamp will lightly grip the guidewire and be pulled into the ramp of the main body of the device [9]. On contact with the ramp feature of the main body of the device, the radius of the main body of the ramp feature and the clamp component combine to further close the central hole of the clamp. This closing effect tightens the grip of the clamp on the guidewire and stops the movement of the guidewire in the inward direction [10].

The Luer hub [3] component is preferably manufactured by injection moulding a plastics material. Preferably the plastics material is selected from polypropylene, polycarbonate or ABS. Preferably, it has a length of about 11 mm and a maximum outside diameter of about 8 mm is bonded to the main body component by either press fit retention, or using bonding adhesives. At its lower end, the component has a hole diameter of about 1 mm to about 2 mm, which is greater than that of the guide wire which allows for free unrestricted movement of the guide wire through the component.

The Luer hub component has an internal taper feature (which conforms to syringe manufacturing standards) that allows connection to a standard syringe, and a locking screw feature on the top of the upper end of the component (conforming to syringe manufacturing standards) which allows a syringe to be locked to the component. It is intentional that the Luer Hub component is compatible with both locking and slip style syringes.

The locking sleeve [4] component is preferably manufactured by injection moulding a plastics material. Preferably, the plastics material is selected from polypropylene, polycarbonate or ABS. Preferably, it has a length of about 12.7 mm and a maximum outside diameter of about 11 mm is press fitted on to the main body component, and is retained by a barb feature which is present on the main body component. The locking sleeve is able to move freely in a rotational direction around the main body allowing for securing to the catheter without causing rotation movement of the assembly. There is an internal thread present in the internal bore of the locking sleeve (manufactured in line with syringe manufacturing standards) which allows for secure screw connection to the catheter.

The device defines at least one lumen which extends through the main body component, Luer hub component and locking sleeve component for allowing flushing fluid through the device. The lumen allows free passage of fluids through the device. This provides the advantage of enabling a catheter with the device attached to be used to administer fluids, or drain effluent. In addition, in the event that an operator forgets to remove the device once a catheter or drain has been put in place it provides the advantage of mitigating risk to the patient.

The lumen comprises a plurality of fenestrations between the clamp and an inner wall of the main body.

Advantageously, the clamp effectively locks the guidewire even if the guidewire or clamp are coated in saline, blood or other body fluids.

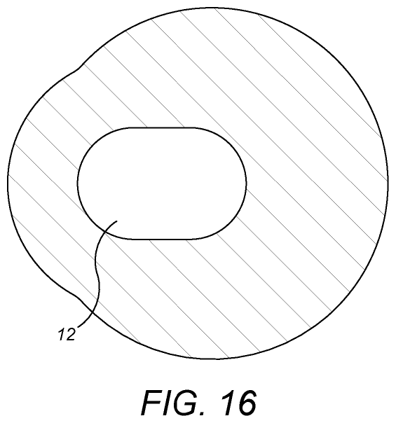

With reference to FIGS. 15 and 16, in a fifth embodiment the guide wire [5] is restricted from moving in the inwards direction by progressively increasing the clamping force a ball applies on the guide wire via a taper activation method.

The device is designed to function to its intended use in all orientations including, upright, lay flat or inverted, positions as well as any other orientation in between. As an assembled product, the device is about 31 mm in length and securely connects to the Luer hub of a standard Catheter. The device has a central lumen which allows free and unrestricted movement of a guidewire through the centre of the device in one direction (free movement), but automatically grips and locks the guidewire in place preventing movement of the guidewire through the device in the opposite direction (lock). This gripping effect automatically releases on changing the direction of the guidewire movement to outwards. In both the configurations of the device (free movement or locked), the design of the device enables bidirectional free flow of fluid through the device.

The main body [1] component is preferably manufactured by injection moulding, or ultrasonic welding, of plastics materials such as ABS, polypropylene or polycarbonate. Preferably, the main body component is colourless or clear. Preferably, it has a length of about 22 mm and a maximum outside diameter of about 8 mm. The main body component has an internal empty volume [6] of about 38.88 mm³ which allows the user to have full visibility of the guidewire and procedural fluids. As shown in FIG. 16, preferably the main body [1] component defines a single channel in which the guidewire and clamp ball are located. In contrast to the first embodiment described above, this embodiment includes a single channel which receives both the guide wire and the clamp ball. The main body component also has an internal shallow radius [7] of about 32 mm that forms the ramp feature necessary to activate the clamp ball component. At the lower end of the main body, the shallow external inclusive angle of about 3.4° of the outside profile is manufactured to provide a secure leak free taper fit to the mating catheter.

The clamp ball [2] component is manufactured plastic materials such as polypropylene, polycarbonate or ABS, or stainless 304 and 316 materials, and is housed inside the main body of the component, where it will be free to move in a forward and backward direction along the ramp feature in the main body during use. Preferably, a single clamp ball is provided. The clamp ball has a diameter configured so that it remains in contact with the guide wire in both the lock and unlock modes.

When the guidewire is pushed or pulled through the male component on the Luer hub in an outwards direction (away from the patient) it is directed through the central hole of the main body. The clamp ball lightly grips the guidewire via its critically controlled outside diameter but does not provide any resistance to wire movement [8]. If the guidewire is pushed in an inwards direction (towards the patient), the critically controlled outside diameter means that the clamp ball will lightly grip the guidewire and be pulled into the ramp of the main body of the device. On contact with the ramp feature of the main body of the device, the shallow radius of the main body of the ramp feature and the clamp ball component combine to tighten the grip of the clamp ball on the guidewire and this stops the movement of the guidewire in the inward direction [9].

The Luer hub [3] component is preferably manufactured by injection moulding a plastics material. Preferably the plastics material is selected from polypropylene, polycarbonate or ABS. Preferably, it has a length of about 11 mm and a maximum outside diameter of about 8 mm is bonded to the main body component by either press fit retention, or using bonding adhesives. At its lower end, the component has a hole diameter of about 1 mm to about 2 mm, which is greater than that of the guide wire which allows for free unrestricted movement of the guide wire through the component. The Luer hub component has an internal taper feature (which conforms to syringe manufacturing standards) that allows connection to a standard syringe, and a locking screw feature on the top of the upper end of the component (conforming to syringe manufacturing standards) which allows a syringe to be locked to the component. It is intentional that the Luer Hub component is compatible with both locking and slips style syringes.

The locking sleeve [4] component is preferably manufactured by injection moulding a plastics material. Preferably, the plastics material is selected from polypropylene, polycarbonate or ABS. Preferably, it has a length of about 11 mm and a maximum outside diameter of about 12.7 mm is press fitted on to the main body component, and is retained by a barb feature which is present on the main body component. The locking sleeve is able to move freely in a rotational direction around the main body allowing for securing to the catheter without causing rotation movement of the assembly. There is an internal thread present in the internal bore of the locking sleeve (manufactured in line with syringe manufacturing standards) which allows for secure screw connection to the catheter.

The device defines at least one lumen which extends through the main body component, Luer hub component and locking sleeve component for allowing flushing fluid through the device. The lumen allows free passage of fluids through the device. This provides the advantage of enabling a catheter with the device attached to be used to administer fluids, or drain effluent. In addition, in the event that an operator forgets to remove the device once a catheter or drain has been put in place it provides the advantage of mitigating risk to the patient.

Advantageously, the clamp ball effectively locks the guidewire even if the guidewire or clamp ball are coated in saline, blood or other body fluids.

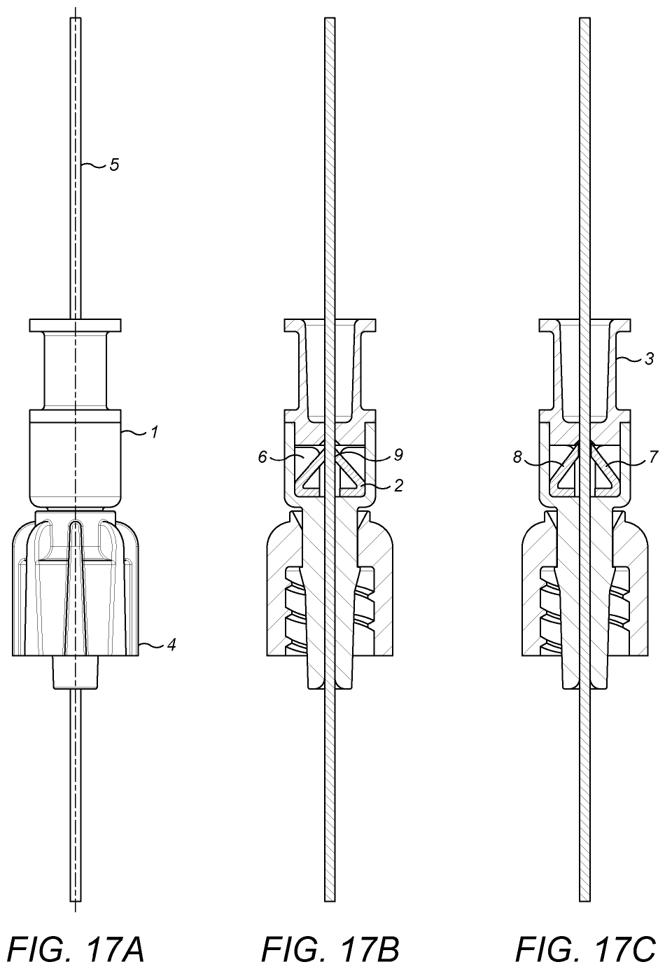

With reference to FIG. 17, in a sixth embodiment the guide wire [5] is restricted from moving in the inwards direction by the pre-formed protruding grip washer arms biting in to the guide wire.

The device is designed to function to its intended use in all orientations including, upright, lay flat or inverted, positions as well as any other orientation in between. As an assembled product, the device is about 32.7 mm in length and securely connects to the Luer hub of a standard Catheter. The device has a central lumen which allows free and unrestricted movement of a guidewire through the centre of the device in one direction (free movement), but automatically grips and locks the guidewire in place preventing movement of the guidewire through the device in the opposite direction (lock). This gripping effect automatically releases on changing the direction of the guidewire movement to outwards. In both the configurations of the clamp (free movement or locked), the design of the device enables bidirectional free flow of fluid through the device.

The main body [1] component is preferably manufactured by injection moulding, or ultrasonic welding, of plastics materials such as ABS, polypropylene or polycarbonate. Preferably, the main body component is colourless or clear. Preferably, it has a length of about 23.7 mm and a maximum outside diameter of about 8 mm. The main body component has an internal empty volume of about 125.9 mm³ [6] which allows the user to have full visibility of the guidewire and procedural fluids. At the lower end of the main body, the shallow external angle of about 3.4° of the outside profile is manufactured to provide a secure leak free taper fit to the mating catheter.

The grip washer [2] component is manufactured by stamping and forming metal materials such as stainless steel grades 304 and 316, and is securely housed through either press fit or adhesive bonding in a specific orientation inside the main body of the component. The grip washer has a height of about 4.2 mm and a maximum outside diameter of about 6 mm and has at least 2 leg features equally spaced in a round construction, separated by empty spaces and connected to each other at the larger diameter end of the component. The angle of the leg features [7] are critically controlled to create a gap at the small end which is less than that of the diameter of the specific guidewire. The width of the leg features of the grip washer is finely tuned to act as a spring on the guidewire and have a gap of about 0.2 mm. The large diameter end of the clamp component has a hole diameter of about 2 mm which is greater than that of the guide wire and allows for free unrestricted movement of the guide wire through this section of the component.

When the guidewire is pushed or pulled through the male component on the Luer hub in an outwards direction (away from the patient) it is directed through the central hole of the grip washer. The clamp legs lightly grips the guidewire via its finely tuned leg widths but does not provide any resistance to wire movement [8]. If the guidewire is pushed in an inwards direction (towards the patient), the finely tuned leg features means that the clamp will lightly bite in to the guidewire. This biting effect tightens the grip of the grip washer on the guidewire and stops the movement of the guidewire in the inward direction [9].

The Luer hub [3] component is preferably manufactured by injection moulding a plastics material. Preferably the plastics material is selected from polypropylene, polycarbonate or ABS. Preferably, it has a length of about 15.25 mm and a maximum outside diameter of about 8 mm is bonded to the main body component by either press fit retention, or using bonding adhesives. At its lower end, the component has a hole diameter of about 1 mm to about 2 mm, which is greater than that of the guide wire which allows for free unrestricted movement of the guide wire through the component. The Luer hub component has an internal taper feature (which conforms to syringe manufacturing standards) that allows connection to a standard syringe, and a locking screw feature on the top of the upper end of the component (conforming to syringe manufacturing standards) which allows a syringe to be locked to the component. It is intentional that the Luer Hub components is compatible with both locking and slip style syringes.

The locking sleeve [4] component is preferably manufactured by injection moulding a plastics material. Preferably, the plastics material is selected from polypropylene, polycarbonate or ABS. Preferably, it has a length of about 12.7 mm and a maximum outside diameter of about 11 mm is press fitted on to the main body component, and is retained by a barb feature which is present on the main body component. The locking sleeve is able to move freely in a rotational direction around the main body allowing for securing to the catheter without causing rotation movement of the assembly. There is an internal thread present in the internal bore of the locking sleeve (manufactured in line with syringe manufacturing standards) which allows for secure screw connection to the catheter.

The device defines at least one lumen which extends through the main body component, Luer hub component and locking sleeve component for allowing flushing fluid through the device. The lumen allows free passage of fluids through the device. This provides the advantage of enabling a catheter with the device attached to be used to administer fluids, or drain effluent. In addition, in the event that an operator forgets to remove the device once a catheter or drain has been put in place it provides the advantage of mitigating risk to the patient.

Advantageously, the grip washer effectively locks the guidewire even if the guidewire or grip washer are coated in saline, blood or other body fluids.

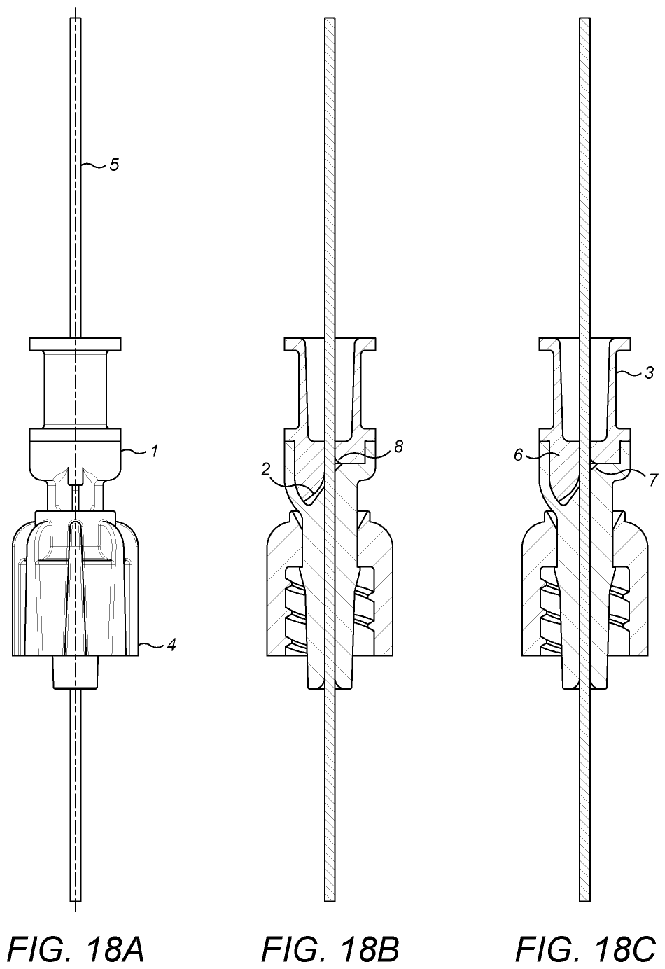

With reference to FIG. 18, in a seventh embodiment the guide wire [5] is restricted from moving in the inwards direction by the stainless steel insert biting in to the guide wire.

The device is designed to function to its intended use in all orientations including, upright, lay flat or inverted, positions as well as any other orientation in between. As an assembled product, the device is about 30.2 mm in length and securely connects to the Luer hub of a standard Catheter. The device has a central lumen which allows free and unrestricted movement of a guidewire through the centre of the device in one direction (free movement), but automatically grips and locks the guidewire in place preventing movement of the guidewire through the device in the opposite direction (lock). This gripping effect automatically releases on changing the direction of the guidewire movement to outwards. In both the configurations of the clamp (free movement or locked), the design of the device enables bidirectional free flow of fluid through the device.

The main body [1] component is preferably manufactured by injection moulding, or ultrasonic welding, of plastics materials such as ABS, polypropylene or polycarbonate. Preferably, the main body component is colourless or clear. Preferably, it has a length of about 21.2 mm and a maximum outside diameter of about 8 mm. The main body component has an internal empty volume [6] of about 23.75 mm³ which allows the user to have full visibility of the guidewire and procedural fluids. At the lower end of the main body, the shallow external inclusive angle of about 3.4° of the outside profile is manufactured to provide a secure leak free taper fit to the mating catheter.

The steel [2] insert component is manufactured by stamping and forming metal materials such as stainless steel grades 304 and 316, and is securely housed through either press fit of adhesive bonding in a specific orientation inside the main body of the component. The steel insert has a height of about 3 mm and a component width of about 1.5 mm. The angle of the steel insert is critically controlled to create a gap between the main body component and steel insert which is less than that of the diameter of the specific guidewire. The material thickness (about 0.5 mm) of the leg features of the steel insert is finely tuned to act as a spring on the guidewire.

When the guidewire is pushed or pulled through the male component on the Luer hub in an outwards direction (away from the patient) it is directed through the gap feature between the steel insert and the main body. The steel insert lightly grips the guidewire via its finely tuned material thickness but does not provide any resistance to wire movement [7]. If the guidewire is pushed in an inwards direction towards the patient, then the finely tuned steel insert will lightly bite in to the guidewire. This biting effect tightens the grip of the steel insert on the guidewire and stops the movement of the guidewire in the inward direction [8].

The Luer hub [3] component is preferably manufactured by injection moulding a plastics material. Preferably the plastics material is selected from polypropylene, polycarbonate or ABS. Preferably, it has a length of about 13.7 mm and a maximum outside diameter of about 8 mm is bonded to the main body component by either press fit retention, or using bonding adhesives. At its lower end, the component has a hole diameter of about 1 mm to about 2 mm, which is greater than that of the guide wire which allows for free unrestricted movement of the guide wire through the component. The Luer hub component has an internal taper feature (which conforms to syringe manufacturing standards) that allows connection to a standard syringe, and a locking screw feature on the top of the upper end of the component (conforming to syringe manufacturing standards) which allows a syringe to be locked to the component. It is intentional that the Luer Hub components is compatible with both locking and slip style syringes.

The locking sleeve [4] component is preferably manufactured by injection moulding a plastics material. Preferably, the plastics material is selected from polypropylene, polycarbonate or ABS. Preferably, it has a length of about 12.7 mm and a maximum outside diameter of about 11 mm is press fitted on to the main body component, and is retained by a barb feature which is present on the main body component. The locking sleeve is able to move freely in a rotational direction around the main body allowing for securing to the catheter without causing rotation movement of the assembly. There is an internal thread present in the internal bore of the locking sleeve (manufactured in line with syringe manufacturing standards) which allows for secure screw connection to the catheter.

The device defines at least one lumen which extends through the main body component, Luer hub component and locking sleeve component for allowing flushing fluid through the device. The lumen allows free passage of fluids through the device. This provides the advantage of enabling a catheter with the device attached to be used to administer fluids, or drain effluent. In addition, in the event that an operator forgets to remove the device once a catheter or drain has been put in place it provides the advantage of mitigating risk to the patient.

Advantageously, the steel insert component effectively locks the guidewire even if the guidewire or steel insert component are coated in saline, blood or other body fluids.

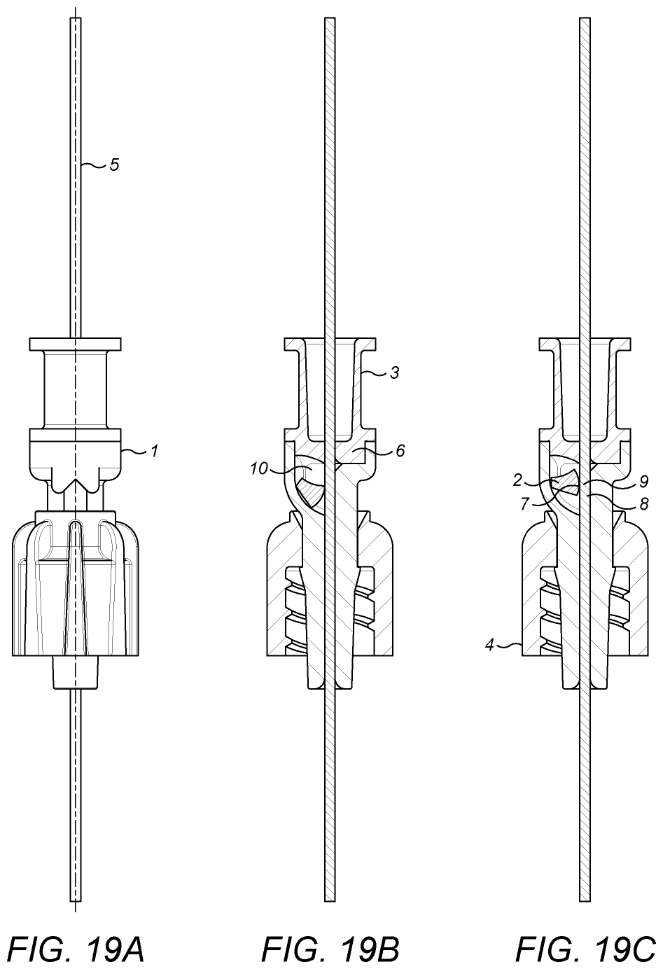

With reference to FIG. 19, in an eighth seventh embodiment the guide wire [5] is restricted from moving in the inwards direction by progressively increasing the clamping force the cam lock applies on the guide wire via an involute form moulded on to the sprung Cam.

The device is designed to function to its intended use in all orientations including, upright, lay flat or inverted, positions as well as any other orientation in between. As an assembled product, the device is about 30.2 mm in length and securely connects to the Luer hub of a standard Catheter. The device has a central lumen which allows free and unrestricted movement of a guidewire through the centre of the device in one direction (free movement), but automatically grips and locks the guidewire in place preventing movement of the guidewire through the device in the opposite direction (lock). This gripping effect automatically releases on changing the direction of the guidewire movement to outwards. In both the configurations of the device (free movement or locked), the design of the device enables bidirectional free flow of fluid through the device.

The main body [1] component is preferably manufactured by injection moulding, or ultrasonic welding, of plastics materials such as ABS, polypropylene or polycarbonate. Preferably, the main body component is colourless or clear. Preferably, it has a length of about 21.2 mm and a maximum outside diameter of about 8 mm. The main body component has an internal empty volume [6] of about 40.5 mm³ which allows the user to have full visibility of the guidewire and procedural fluids. The main body component also has an internal radius [7] of about 3.2 mm that forms the spring housing feature necessary to activate the cam component. At the lower end of the main body, the shallow external inclusive angle of about 3.4° of the outside profile is manufactured to provide a secure leak free taper fit to the mating catheter.

The cam [2] component is manufactured plastic materials such as polypropylene, polycarbonate and ABS, and is housed inside the main body of the component, where it is attached by bonding adhesive or press fit in to the internal radius of the main body. Once situated in place, the cam will be free to move in a forward and backward radial motion pivoting from the top of the spring section of the cam. This forward and backward pivoting motion, coupled with the involute radius [8] shape of about 1.4 mm situated on the front of the cam has the effect of adjusting the through hole diameter in which the guide wire will run through during use.

When the guidewire is pushed or pulled through the male component on the Luer hub in an outwards direction (away from the patient) it is directed through the central hole of the main body. The cam component lightly grips [9] the guidewire on the involute radius via its critically controlled spring pressure, but does not provide any resistance to wire movement. If the guidewire is pushed in an inwards direction (towards the patient), the involute radius of the cam means that the it will lightly grip the guidewire and be pulled in a downward rotating motion, where the increasing diameter of the involute radius and the pivot feature of the component combine to tighten the grip [10] of the cam on the guidewire and stops the movement of the guidewire in the inward direction.

The Luer hub [3] component is preferably manufactured by injection moulding a plastics material. Preferably the plastics material is selected from polypropylene, polycarbonate or ABS. Preferably, it has a length of about 12.7 mm and a maximum outside diameter of about 8 mm is bonded to the main body component by either press fit retention, or using bonding adhesives. At its lower end, the component has a hole diameter of about 1 mm to about 2 mm, which is greater than that of the guide wire which allows for free unrestricted movement of the guide wire through the component. The Luer hub component has an internal taper feature (which conforms to syringe manufacturing standards) that allows connection to a standard syringe, and a locking screw feature on the top of the upper end of the component (conforming to syringe manufacturing standards) which allows a syringe to be locked to the component. It is intentional that the Luer Hub component is compatible with both locking and slips style syringes.

The locking sleeve [4] component is preferably manufactured by injection moulding a plastics material. Preferably, the plastics material is selected from polypropylene, polycarbonate or ABS. Preferably, it has a length of about 12.7 mm and a maximum outside diameter of about 11 mm is press fitted on to the main body component, and is retained by a barb feature which is present on the main body component. The locking sleeve is able to move freely in a rotational direction around the main body allowing for securing to the catheter without causing rotation movement of the assembly. There is an internal thread present in the internal bore of the locking sleeve (manufactured in line with syringe manufacturing standards) which allows for secure screw connection to the catheter.

The device defines at least one lumen which extends through the main body component, Luer hub component and locking sleeve component for allowing flushing fluid through the device. The lumen allows free passage of fluids through the device. This provides the advantage of enabling a catheter with the device attached to be used to administer fluids, or drain effluent. In addition, in the event that an operator forgets to remove the device once a catheter or drain has been put in place it provides the advantage of mitigating risk to the patient.

Advantageously, the cam component effectively locks the guidewire even if the guidewire or cam component are coated in saline, blood or other body fluids.

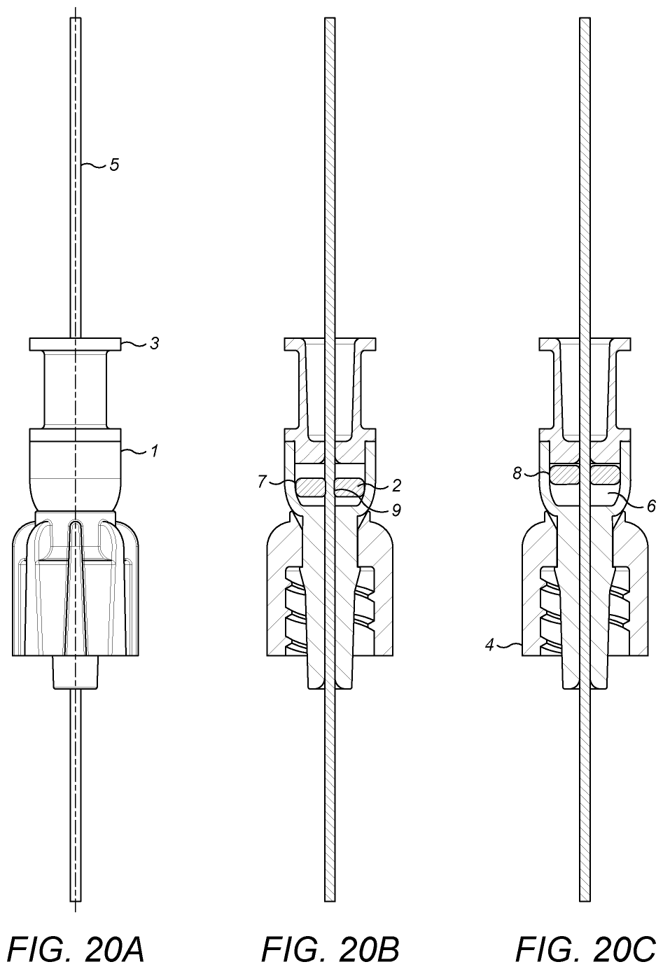

With reference to FIG. 20, in a ninth embodiment the guide [5] wire is restricted from moving in the inwards direction by progressively increasing the compression force acting on the guide wire via a taper activation method.

The device is designed to function to its intended use in all orientations including, upright, lay flat or inverted, positions as well as any other orientation in between. As an assembled product, the device is about 31 mm in length and securely connects to the Luer hub of a standard Catheter. The device has a central lumen which allows free and unrestricted movement of a guidewire through the centre of the device in one direction (free movement), but automatically grips and locks the guidewire in place preventing movement of the guidewire through the device in the opposite direction (lock). This gripping effect automatically releases on changing the direction of the guidewire movement to outwards. In both the configurations of the O ring (free movement or locked), the design of the device enables bidirectional free flow of fluid through the device.

The main body [1] component is preferably manufactured by injection moulding, or ultrasonic welding, of plastics materials such as ABS, polypropylene or polycarbonate. Preferably, the main body component is colourless or clear. Preferably, it has a length of about 22 mm and a maximum outside diameter of about 8 mm. The main body component has an internal empty volume [6] of about 85.3 mm³ which allows the user to have full visibility of the guidewire and procedural fluids. The main body component also has an internal shallow inclusive angle [7] of about 4° that forms the ramp feature necessary to activate the O ring component. At the lower end of the main body, the shallow external inclusive angle about 3.4° of the outside profile is manufactured to provide a secure leak free taper fit to the mating catheter.

The O ring [2] component is manufactured by injection moulding plastic materials such as Nitrile, TPU and PVC, has a critically controlled shore rating of plastic (70 Shore to Shore), and is housed in a specific orientation inside the main body of the component, where it will be free to move in a forward and backward direction during use. The O ring has a height of about 2 mm and a maximum outside diameter of about 6.1 mm. A hole of equal or smaller diameter to the specific guidewire and a diameter of about 0.5 mm is present through the centre of the component. The outside profile has radius features on both diameter edges of about 0.8 mm that contact the internal angle of the main body (ramp).

When the guidewire is pushed or pulled through the male component on the Luer hub in an outwards direction (away from the patient) it is directed through the central hole of the O ring. The O ring lightly grips [8] the guidewire via its critically controlled central hole but does not provide any resistance to wire movement. If the guidewire is pushed in an inwards direction (towards the patient), the critically controlled central hole means that the O ring will lightly grip the guidewire and be pulled into the ramp of the main body of the device. On contact with the ramp feature of the main body of the device, the angles of the main body of the ramp feature and the O ring component combine to compress the O ring material which further closes the central hole of the O ring. This compressing effect tightens the grip of the O ring [9] on the guidewire and stops the movement of the guidewire in the inward direction.

The Luer hub [3] component is preferably manufactured by injection moulding a plastics material. Preferably the plastics material is selected from polypropylene, polycarbonate or ABS. Preferably, it has a length of about 11 mm and a maximum outside diameter of about 8 mm is bonded to the main body component by either press fit retention, or using bonding adhesives. At its lower end, the component has a hole diameter of about 1 mm to about 2 mm, which is greater than that of the guide wire which allows for free unrestricted movement of the guide wire through the component. The Luer hub component has an internal taper feature (which conforms to syringe manufacturing standards) that allows connection to a standard syringe, and a locking screw feature on the top of the upper end of the component (conforming to syringe manufacturing standards) which allows a syringe to be locked to the component. It is intentional that the Luer Hub component is compatible with both locking and slips style syringes.

The locking sleeve [4] component is preferably manufactured by injection moulding a plastics material. Preferably, the plastics material is selected from polypropylene, polycarbonate or ABS. Preferably, it has a length of about 12.7 mm and a maximum outside diameter of about 11 mm is press fitted on to the main body component, and is retained by a barb feature which is present on the main body component. The locking sleeve is able to move freely in a rotational direction around the main body allowing for securing to the catheter without causing rotation movement of the assembly. There is an internal thread present in the internal bore of the locking sleeve (manufactured in line with syringe manufacturing standards) which allows for secure screw connection to the catheter.

The device defines at least one lumen which extends through the main body component, Luer hub component and locking sleeve component for allowing flushing fluid through the device. The lumen allows free passage of fluids through the device. This provides the advantage of enabling a catheter with the device attached to be used to administer fluids, or drain effluent. In addition, in the event that an operator forgets to remove the device once a catheter or drain has been put in place it provides the advantage of mitigating risk to the patient.

Advantageously, the O ring component effectively locks the guidewire even if the guidewire or O ring component are coated in saline, blood or other body fluids.

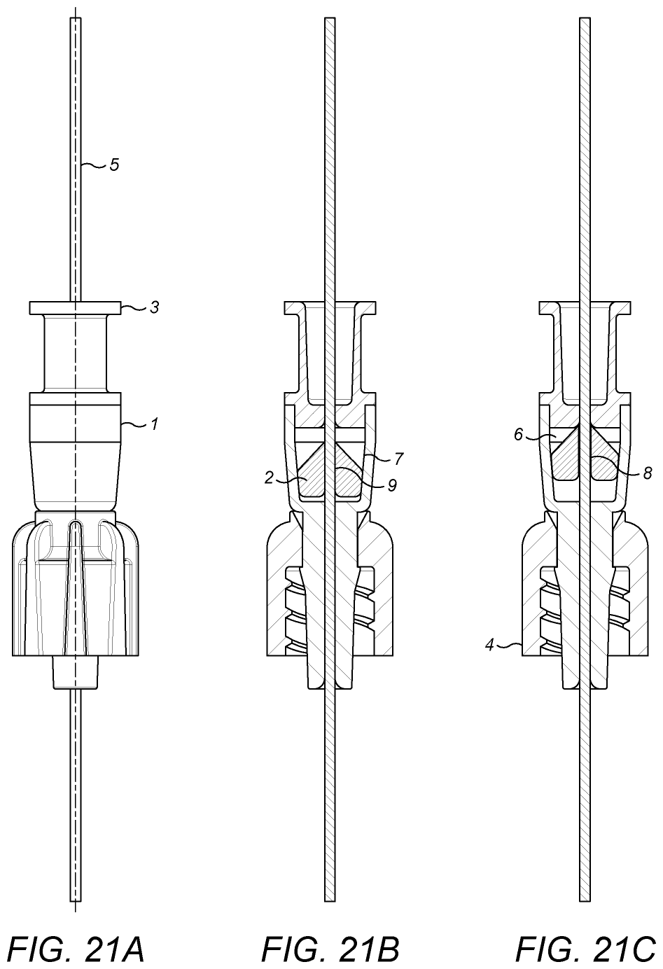

With reference to FIG. 21, in a tenth embodiment the guide wire [5] is restricted from moving in the inwards direction by progressively increasing the compression force acting on the guide wire via a duckbill and taper activation method.

The device is designed to function to its intended use in all orientations including, upright, lay flat or inverted, positions as well as any other orientation in between. As an assembled product, the device is about 34 mm in length and securely connects to the Luer hub of a standard catheter. The device has a central lumen which allows free and unrestricted movement of a guidewire through the centre of the device in one direction (free movement), but automatically grips and locks the guidewire in place preventing movement of the guidewire through the device in the opposite direction (lock). This gripping effect automatically releases on changing the direction of the guidewire movement to outwards. In both the configurations of the duckbill (free movement or locked), the design of the device enables bidirectional free flow of fluid through the device.

The main body [1] component is preferably manufactured by injection moulding, or ultrasonic welding, of plastics materials such as ABS, polypropylene or polycarbonate. Preferably, the main body component is colourless or clear. Preferably, it has a length of about 25 mm and a maximum outside diameter of about 8 mm. The main body component has an internal empty volume [6] of about 121 mm³ which allows the user to have full visibility of the guidewire and procedural fluids. The main body component also has an internal shallow angle [7] of about 4° that forms the ramp feature necessary to activate the Duckbill component. At the lower end of the main body, the shallow external inclusive angle about 3.4° of the outside profile is manufactured to provide a secure leak free taper fit to the mating catheter.

The duckbill [2] component is manufactured by injection moulding plastic materials such as Nitrile, TPU and PVC, has a critically controlled shore rating of plastic (70 Shore to 90 Shore), and is housed in a specific orientation inside the main body of the component, where it will be free to move in a forward and backward direction during use. The duckbill has a height of about 5 mm and a maximum outside diameter of about 6.1 mm. A gap of equal or smaller diameter to the specific guidewire and a distance of about 0.1 mm is present through the centre of the component. The outside profile has an equal or slightly greater angle of about 80° to that of the internal angle of the main body (ramp).

When the guidewire is pushed or pulled through the male component on the Luer hub in an outwards direction (away from the patient) it is directed through the central hole of the Duckbill. The duckbill lightly grips [8] the guidewire via its critically controlled central hole but does not provide any resistance to wire movement. If the guidewire is pushed in an inwards direction (towards the patient), the critically controlled central hole means that the duckbill will lightly grip the guidewire and be pulled into the ramp of the main body of the device. On contact with the ramp feature of the main body of the device, the angles of the main body of the ramp feature and the duckbill component combines to compress the duckbill material which further closes a central channel through the duckbill. This compressing effect tightens the grip of the duckbill on the guidewire [9] and stops the movement of the guidewire in the inward direction.

The Luer hub [3] component is preferably manufactured by injection moulding a plastics material. Preferably the plastics material is selected from polypropylene, polycarbonate or ABS. Preferably, it has a length of about 11 mm and a maximum outside diameter of about 8 mm is bonded to the main body component by either press fit retention, or using bonding adhesives. At its lower end, the component has a hole diameter of about 1 mm to about 2 mm, which is greater than that of the guide wire which allows for free unrestricted movement of the guide wire through the component. The Luer hub component has an internal taper feature (which conforms to syringe manufacturing standards) that allows connection to a standard syringe, and a locking screw feature on the top of the upper end of the component (conforming to syringe manufacturing standards) which allows a syringe to be locked to the component. It is intentional that the Luer Hub component is compatible with both locking and slips style syringes.

The locking sleeve [4] component is preferably manufactured by injection moulding a plastics material. Preferably, the plastics material is selected from polypropylene, polycarbonate or ABS. Preferably, it has a length of about 12.7 mm and a maximum outside diameter of about 11 mm. Preferably, it is press fitted on to the main body component, and is retained by a barb feature which is present on the main body component. The locking sleeve is able to move freely in a rotational direction around the main body allowing for securing to the catheter without causing rotation movement of the assembly. Preferably, there is an internal thread present in the internal bore of the locking sleeve (manufactured in line with syringe manufacturing standards) which allows for secure screw connection to the catheter.

The device defines at least one lumen which extends through the main body component, Luer hub component and locking sleeve component for allowing flushing fluid through the device. The lumen allows free passage of fluids through the device. This provides the advantage of enabling a catheter with the device attached to be used to administer fluids, or drain effluent. In addition, in the event that an operator forgets to remove the device once a catheter or drain has been put in place it provides the advantage of mitigating risk to the patient.

Advantageously, the Duckbill component effectively locks the guidewire even if the guidewire or Duckbill component are coated in saline, blood or other body fluids.

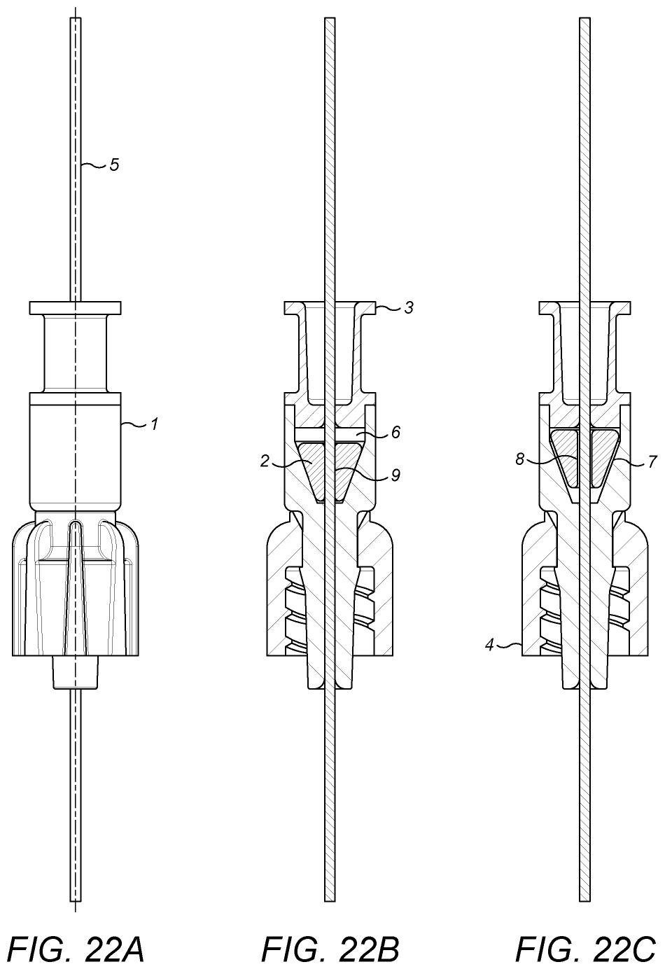

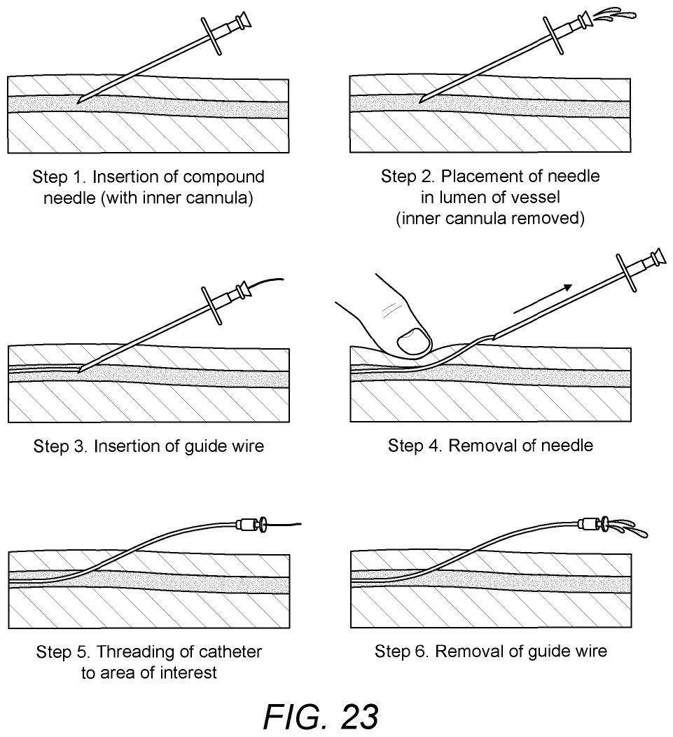

With reference to FIG. 22, in an eleventh embodiment the guide wire [5] is restricted from moving in the inwards direction by progressively increasing the compression force acting on the guide wire via wedge and a taper activation method.

The device is designed to function to its intended use in all orientations including, upright, lay flat or inverted, positions as well as any other orientation in between. As an assembled product, the device is about 34 mm in length and securely connects to the Luer hub of a standard Catheter. The device has a central lumen which allows free and unrestricted movement of a guidewire through the centre of the device in one direction (free movement), but automatically grips and locks the guidewire in place preventing movement of the guidewire through the device in the opposite direction (lock). This gripping effect automatically releases on changing the direction of the guidewire movement to outwards. In both the configurations of the wedge (free movement or locked), the design of the device enables bidirectional free flow of fluid through the device.

The main body [1] component is preferably manufactured by injection moulding, or ultrasonic welding, of plastics materials such as ABS, polypropylene or polycarbonate. Preferably, the main body component is colourless or clear. Preferably, it has a length of about 25 mm and a maximum outside diameter of about 8 mm. The main body component has an internal empty volume [6] of about 89 mm³ which allows the user to have full visibility of the guidewire and procedural fluids. The main body component also has an internal shallow inclusive angle [7] of about 40° that forms the ramp feature necessary to activate the Wedge component. At the lower end of the main body, the shallow external inclusive angle about 3.4° of the outside profile is manufactured to provide a secure leak free taper fit to the mating catheter.

The Wedge [2] component is manufactured by injection moulding plastic materials such as Nitrile, TPU and PVC, has a critically controlled shore rating of plastic (70 Shore to 90 Shore), and is housed in a specific orientation inside the main body of the component, where it will be free to move in a forward and backward direction during use. The Wedge has a height of about 5 mm and a maximum outside diameter of about 6 mm. A gap of equal or smaller in diameter to the specific guidewire and a distance of about 0.2 mm is present through the centre of the component. The outside profile has an equal or slightly greater inclusive angle of about 40° to that of the internal angle of the main body (ramp).

When the guidewire is pushed or pulled through the male component on the Luer hub in an outwards direction (away from the patient) it is directed through the central hole of the Wedge. The Wedge lightly grips the guidewire [8] via its critically controlled central hole but does not provide any resistance to wire movement. If the guidewire is pushed in an inwards direction (towards the patient), the critically controlled central hole means that the Wedge will lightly grip the guidewire and be pulled into the ramp of the main body of the device. On contact with the ramp feature of the main body of the device, the angles of the main body of the ramp feature and the Wedge component combine to compress the Wedge material which further closes the central hole of the Wedge. This compressing effect tightens the grip of the Wedge on the guidewire [9] and stops the movement of the guidewire in the inward direction.

The Luer hub [3] component is preferably manufactured by injection moulding a plastics material. Preferably the plastics material is selected from polypropylene, polycarbonate or ABS. Preferably, it has a length of about 11 mm and a maximum outside diameter of about 8 mm is bonded to the main body component by either press fit retention, or using bonding adhesives. At its lower end, the component has a hole diameter of about 1 mm to about 2 mm, which is greater than that of the guide wire which allows for free unrestricted movement of the guide wire through the component.

The Luer hub component has an internal taper feature (which conforms to syringe manufacturing standards) that allows connection to a standard syringe, and a locking screw feature on the top of the upper end of the component (conforming to syringe manufacturing standards) which allows a syringe to be locked to the component. It is intentional that the Luer Hub component is compatible with both locking and slips style syringes.

The locking sleeve [4] component is preferably manufactured by injection moulding a plastics material. Preferably, the plastics material is selected from polypropylene, polycarbonate or ABS. Preferably, it has a length of about 12.7 mm and a maximum outside diameter of about 11 mm is press fitted on to the main body component, and is retained by a barb feature which is present on the main body component. The locking sleeve is able to move freely in a rotational direction around the main body allowing for securing to the catheter without causing rotation movement of the assembly. There is an internal thread present in the internal bore of the locking sleeve (manufactured in line with syringe manufacturing standards) which allows for secure screw connection to the catheter.

The device defines at least one lumen which extends through the main body component, Luer hub component and locking sleeve component for allowing flushing fluid through the device. The lumen allows free passage of fluids through the device. This provides the advantage of enabling a catheter with the device attached to be used to administer fluids, or drain effluent. In addition, in the event that an operator forgets to remove the device once a catheter or drain has been put in place it provides the advantage of mitigating risk to the patient.

Advantageously, the wedge component effectively locks the guidewire even if the guidewire or wedge component are coated in saline, blood or other body fluids.

The above described embodiments have been given by way of example only, and the skilled reader will naturally appreciate that many variations could be made thereto without departing from the scope of the invention.

### REFERENCES

1) Williams, T L, Bowdle, A T, Winters, B D, Pavkovic, S D, Szekendi, M. Guidewires unintentionally retained during central venous catheterization. J Assoc Vasc Access 2014; 19:29-34
2) Evans, L V, Dodge, K L. Simulation and patient safety: Evaluative checklists for central venous catheter insertion. Qual Saf Health Care 2010; 19(Suppl 3):i42-46
3) Vannucci, A, Jeffcoat, A, Ifune, C, Salinas, C, Duncan, J R, Wall, M. Retained guidewires after intraoperative placement of central venous catheters. Anaesth Analg 2013; 117:102-8
4) NHS England and NHS Improvement. Provisional publication of Never Events reported as occurring between 1 Apr. 2019 and 29 Feb. 2020. Published 30 Mar. 2020.
5) Schummer, W, Schummer, C, Gaser, E, Bartunek, R. Loss of the guide wire: Mishap or blunder? Br J Anaesth 2002; 88:144-6 [Article] [PubMed]
6) Anwari, J S, Imran, S. Retention of central line guide wire. Saudi J Anaesth 2014; 8:443-5 [Article] [PubMed]
7) Pokharel, K, Biswas, B K, Tripathi, M, Subedi, A. Missed central venous guide wires: A systematic analysis of published case reports. Crit Care Med 2015; 43:1745-56 [Article] [PubMed]
8) Mariyaselvam, M, Walters, H, Callan, C, Mathew, K, Jackman, S, Young, P. Guidewire retention: Reported incidence, location and timing of error. Eur J Anaesthesiol 2017; 32(suppl 53):16AP02-12

1. A guide wire safety device, comprising: a main body component located between a Luer hub component for connection to a Luer hub:, and a locking sleeve component for connection to a catheter, wherein the main body component houses a means for gripping a guidewire.

2. The guide wire safety device of claim 1, wherein the means for gripping a guidewire includes a gripper.

3. The guide wire safety device of claim 2, wherein the gripper allows free movement of a guidewire outwards towards the Luer hub component from the locking sleeve component and grips the guidewire to prevent, movement of the guidewire through the device in an opposite direction away from the Luer hub component.

4. The guide wire safety device of claim 2, wherein the gripper is biased to grip a guidewire without requiring operator intervention if the guidewire is pulled or pushed through the device from the Luer hub towards the locking sleeve component.

5. The guide wire safety device of claim 2, wherein the gripper is selected from a collet, one or more barbs, a clamp, a clamp ball, a grip washer, a steel insert, a cam lock, an o-ring, a duckbill and a wedge.

6. The guide wire safety device of claim 1, wherein the main body component is manufactured by injection moulding or ultrasonic welding of a plastics material such as acrylonitrile butadiene styrene (ABS), polypropylene or polycarbonate, which is optionally colourless or clear.

7. The guide wire safety device of any one of the preceding claims claim 1, wherein the main body component has length of about 20 mm to about 25 mm.

8. The guide wire safety device of claim 1, wherein the main body component has a maximum outside diameter of about 8 mm.

9. The guide wire safety device of claim 1, wherein the main body component has an internal empty volume of about 20 mm³ to about 136 mm³ .

10. The guide wire safety device of claim 1, wherein an end of the main body component adjacent to the locking sleeve component has a shallow external inclusive angle about 3.4° of the outside profile.

11. The guide wire safety device of claim 1, wherein the Luer hub component is manufactured by injection moulding plastics materials such as polypropylene, polycarbonate or ABS.

12. The guide wire safety device of claim 1, wherein the Luer hub component has a length of about 10 mm to about 15 mm.

13. The guide wire safety device of claim 1, wherein the Luer hub component has a maximum outside diameter of about 8 mm.

14. The guide wire safety device of claim 1, wherein the Luer hub component is bonded to the main body component by either press fit retention, or using one or more bonding adhesives.

15. The guide wire safety device of claim 1, wherein the Luer hub component has an end for connection to a syringe.

16. The guide wire safety device of claim 1, wherein the Luer hub component has an internal taper feature that allows connection to a standard syringe, and/or a locking screw feature on the top of the Luer hub component which allows a syringe to be screwed to the Luer hub component.

17. The guide wire safety device of claim 1, wherein the Luer hub component has a lumen having a diameter of about 0.8 mm to about 1 mm, which is greater than that of a guide wire which allows for free unrestricted movement of the guide wire through the Luer hub component.

18. The guide wire safety device of claim 1, wherein locking sleeve component is manufactured by injection moulding plastic materials such as polypropylene, polycarbonate or ABS.

19. The guide wire safety device of claim 1, wherein the locking sleeve component has a length of about 10 mm to about 15 mm.

20. The guide wire safety device of any one of the preceding claims claim 1, wherein the locking sleeve component has a maximum outside diameter of about 10 mm to about 12 mm.

21. The guide wire safety device of claim 1, wherein the locking sleeve component is press fitted on to the main body component, and is optionally retained by a barb feature which is present on the main body component.

22. The guide wire safety device of claim 1, wherein the locking sleeve component moves freely in a rotational direction around the main body component.

23. The guide wire safety device of claim 1, wherein an internal thread is present in an internal bore of the locking sleeve component which allows for a secure screw connection to the catheter.

24. The guide wire safety device of claim 2, wherein the gripper effectively locks the guidewire even if the guidewire or gripper are coated in saline, blood or other body fluids.

25. The guide wire safety device of claim 2, wherein the gripper is capable of releasing a lock on the guidewire.

26. The guide wire safety device of any one of the preceding claims claim 1, defining at least one lumen which extends through the main body component, Luer hub component and locking sleeve component for allowing flushing fluid through the device.

27. The guide wire safety device of claim 26, wherein the lumen allows free passage of fluids through the device.

28. The guide wire safety device of claim 26, wherein the lumen comprises a plurality of fenestrations between a gripper and an inner wall of the main body component.
